SEQUENCE LISTING (25 sequences)

SEQ ID NO: 1 - protein, 458 aa, Lactobacillus
MASEDKKSKREHIIHFEDTPSKSLDEVNGSVEVPHNAGFWKTLAAYTGPGILVAVGYMDPGNWITSIAGGASFKYSLLSVILISSLIAMLLQAMAARLGIVTGRDLAQMTRDHTSKAMGGFLWVITELAIMATDIAEIIGSAIALKLLFNMPLIVGIIITTADVLILLLLMRLGFRKIEAVVATLVLVILLVFAYEVILAQPNVPELLKGYLPHADIVTNKSMLYLSLGIVGATVMPHDLFLGSSISQTRKIDRTKHEEVKKAIKFSTIDSNLQLTMAFIVNSLLLILGAALFFGTSSSVGRFVDLFNALSNSQIVGAIASPMLSMLFAVALLASGQSSTITGTLAGQIIMEGFIHLKMPLWAQRLLTRLMSVTPVLIFAIYYHGNEAKIENLLTFSQVFLSIALPFAVIPLVLYTSDKKIMGEFANRAWVKWTAWFISGVLIILNLYLIAQTLGFVK

SEQ ID NO: 2 - protein, 450 aa, Lactobacillus
MARPDERLTVQREKRSLDDINRSVQVPSVYESSFFQKFLAYSGPGALVAVGYMDPGNWLTALEGGSRYHYALLSVLLMSILVAMFMQTLAIKLGVVARLDLAQAIAAFIPNWSRICLWLINEAAMMATDMTGVVGTAIALKLLFGLPLMWGMLLTIADVLVVLLFLRFGIRRIELIVLVSILTVGIIFGIEVARADPSIGGIAGGFVPHTDILTNHGMLLLSLGIMGATIMPHNIYLHSSLAQSRKYDEHIPAQVTEALRFGKWDSNVHLVAAFLINALLLILGAALFYGVGGHVTAFQGAYNGLKNPMIVGGLASPLMSTLFAFALLITGLISSIASTLAGQIVMEGYLNIRMPLWERRLLTRLVTLIPIMVIGFMIGFSEHNFEQVIVYAQVSLSIALPFTLFPLVALTNRRDLMGIHVNSQLVRWVGYFLTGVITVLNIQLAISVFV

SEQ ID NO: 3 - protein, 535 aa, Lactobacillus
MSDDHKKRHPIKLIQYANGPSLEEINGTVEVPHGKGFWRTLFAYSGPGALVAVGYMDPGNWSTSITGGQNFQYLLISVILMSSLIAMLLQYMAAKLGIVSQMDLAQAIRARTSKKLGIVLWILTELAIMATDIAEVIGAAIALYLLFHIPLVIAVLVTVLDVLVLLLLTKIGFRKIEAIVVALILVILLVFVYQVALSDPNMGALLKGFIPTGETFASSPSINGMSPIQGALGIIGATVMPHNLYLHSAISQTRKIDYKNPDDVAQAVKFSAWDSNIQLSFAFVVNCLLLVMGVAVFKSGAVKDPSFFGLFQALSDSSTLSNGVLIAVAKSGILSILFAVALLASGQNSTITGTLTGQVIMEGFVHMKMPLWARRLVTRIISVIPVIVCVMLTARDTPIQQHEALNTLMNNSQVFLAFALPFSMLPLLMFTNSKVEMGDRFKNTGWVKVLGWISVLGLTGLNLKGLPDSIAGFFGDHPTATQTNMANIIAIVLIVAILALLAWTIWDLYKGNQRYEAHLAAVADEKEAKADVDEQ

SEQ ID NO: 4 - protein, 458 aa, Lactobacillus casei
MASEDKKSKREHIIHFEDTPSKSLDEVNGSVEVPHNAGFWKTLAAYTVPGILVAVGYMDPGNWITSIAGGASFKYSLLSVILISSLIAMLLQAMAARLGIVTGRDLAQMTRDHTSKAMGGFLWVITELAIMATDIAEIIGSAIALKLLFNMPLIVGIIITTADVLILLLLMRLGFRKIEAVVATLVLVILLVFAYEVILAQPNVPELLKGYLPHADIVTNKSMLYLSLGIVGATVMPHDLFLGSSISQTRKIDRTKHEEVKKAIKFSTIDSNLQLTMAFIVNSLLLILGAALFFGTSSSVGRFVDLFNALSNSQIVGAIASPMLSMLFAVALLASGQSSTITGTLAGQIIMEGFIHLKMPLWAQRLLTRLMSVTPVLIFAIYYHGNEAKIENLLTFSQVFLSIALPFAVIPLVLYTSDKKIMGEFANRAWVKWTAWFISGVLIILNLYLIAQTLGFVK

SEQ ID NO: 5 - protein, 459 aa, Lactobacillus brevis
MKNHETDTKTKHHMIESTGSGQKSLDEVNGTVEVPQNAGFWRTLMAYTGPGALIAVGYMDPGNWITSIAGGAQYKYTLLTVVLLSSLVAMLLQAMSARLGIVTGKDLAQLTREHTGKRTGFALWIITELAIMATDIAEIIGSAIALKLLFGFPLIVGIIITAMDVLVLLVLMKLGFRKIEAIVATLVAVILFVFLYEVILAQPHMGEVLKGYLPSSTVVTNHGMLYLSLGIVGATVMPHDLYLGSSISQTRSFDRKNRKSVAQAIKFTTIDSNIQLTLAFVVNSLLLILGAALFFGTNSDLGRFVDLFNALSDSQIVGAIASPMLSMLFALALLSSGQSSTITGTLAGQIIMEGFINLKMPLWAQRLITRLLSVTPVIIFAIIYHGNEAKIEDLLTFSQVFLSIALPFAMIPLVIFTSSKKLMGEFANRTWSKILGWIIAVILIILNIYLILNTLHIVQ

SEQ ID NO: 6 - protein, 447 aa, Pediococcus acidilactici
MSKKLDEVDNKSLDEINGSIKVPKNAGFFKTLMAYTGPGILIAVGYMDPGNWITSIAGGAQFKYTLLSVVLISSLIAMLLQAMSARLGIVTGKDLAQLTRERTSKRVGFMLWVVAELAIMATDIAEIIGSGIALELLFHIPLIIGILITAADVLILLLLMRLGFRKIEAIVATLVMVILIVFAYEVFLSDPSISGIIKGYVPAPVILQNNSMLYLSLGIVGATVMPHDLYLGSSISQTREIDRRDRKNVAQAIRFSTIDSNMQLFLAFIVNSLLLILGAALFYGTDSSLGRFVDLFNALSDNQIVGAIASPMLSMLFAVALLASGQSSTITGTLSGQIIMEGFIRLRVPLWVQRLVTRLLSVAPVLIFAIYYHGDEAKIENLLTFSQVFLSVALPFAVIPLVMYTSSKKLMGEFANRQWVKWCAWIATIILILLNIYLILQTLGIVK

SEQ ID NO: 7 - protein, 464 aa, Lactobacillus plantarum
MKSAKTKDHAKMKAAEEKAIHSTGADSKSLDEVNGSVRVPKDASFWRTLIAYTGPGALVAVGYMDPGNWITSIAGGSQYKYALLSVILLSSLIAMLLQAMAARLGIVTGKDLAQLTRERTSKGMGIFLWIITELAIMATDVAEIIGSGIALKLLFGFPLIVGILITTADVLILLLLMKLGFRKIEAIVATLVAVILFVFLYEVIISQPNIPEMLKGYVPTSRIVSNRSMLFLALGIVGATVMPHNLYLGSSISQTRQVDRSDEKEVAKAVKFTTIDSNIQLSVAFVVNSLLLILGAALFFGTKGDLGRFVDLYNALGDSKVVGSIASPLLSMLFAIALLSSGQSSTITGTLSGQIIMEGFIRLKMPLWAQRLLTRLISVTPVLAFAIYYHGNEAKIEDLLTMSQVFLSIALPFAMIPLVMFTSNRALMGNFTNRVWVKWTAWIVTVILIILNIYLILQTVGLVK

SEQ ID NO: 8 - protein, 443 aa, Lactobacillus sakei
MHYADGSSLEEINNTVAIPKNAGFWKTLMAFMGPGALVAVGYMDPGNWITSIAGGAQFAYTLISVILVSNLIAMLLQAMAARLGIVTGMDLAQMTRAKTGKKMGIFLWIVTELAIMATDIAEIIGSAIALELIFNIPLLWGVLITAFDVLLLLLLMKLGFRKIEAIVATLVAVILFVFLYEVILAQPNMGDVVRGFVPSPRIMTDKKMLFLALGIVGATVMPHNLYLHSSIAQARQYDRDDVAEKRKAIKFTVIDSNIQLTIAFVVNCLLLILGAAMFYGTNSDLGRFVDLFNALQNKEIVGSIASPMLSLLFAVALLASGQNSTITGTLSGQIVMEGFVRMKIPLWARRVITRGLSILPVIIFTVYYHGNEAQVENLLIYSQVFLSIALPVSMIPLTLFTSDEKIMGPFVNRPWVKYTAWFVTIVLTLLNIYLILQTVGLAA

SEQ ID NO: 9 - protein, 454 aa, Lactobacillus alimentarius
MSSKNKKHESLIHYANGPSLEEINDTVEIPKDAGFFKTLLAYSGPGALVAVGYMDPGNWVTSIAGGAQFKYKLLSVILISSLIAMLLQYMSAKLGIVTGRDLAQLTRDRTSRVGGFILWIITELAIMATDIAEIIGSAIALKLLFNIPVLWGVIITAFDVLLLLVLMKLGFRKIEAIVATLIMVILLVFLYEVILAKPDVGQMMVGFIPEPKILQNQSMLYLSLGIVGATVMPHNLYLHSSISQARKYDRDDPKSIHQAVRFSTWDSNIQLTLAFVVNTLLLLLGAALFYGTSSDLGRFVDLFNALQDPKVAGAVASPVLSILFAVALLASGQNSTITGTLSGQIVMEGFIHMKMKLWARRVITRLMSIIPVITFAIIYHGNEAKIESLLTFSQVFLSVALPFSIFPLIKFTSNKKLMGEFVNNKLVEYIGYFVAIVLTILNIWLIYTTFVPTA

SEQ ID NO: 10 - protein, 451 aa, Lactobacillus floricola
MTKEETKLFHYADGPSLEEINGTVAVPKKGGFWKTLFAFSGPGALVAVGYMDPGNWVTSIAGGAQYQYTLLSVILISSLIAMLLQAMSARLGIASGLDLAQATAKHSPKWLRYTLWIITELAIMATDIAEIVGAAIALKLLFNLPLIVGIFLTTLDVMLLLLLMKLGFRKIEAIVGALIVSILVIFLYEVILARPDVGAMFAGYIPQPEVVTNKGAFYIALGIVGATVMPHNLYLHSSIAQARQYDRNDIEEKKRAIKFTVLDSNIQLSVAFVVNTLLLLLGAALFYGAQTDLGTFSELYNALQNPQVAGVIASPILSVLFAVALLASGQNSTITGTLSGQIVMEGFIHLKMPMWARRVITRLISVIPVLIFAIIYHSNEAKIEDLLVFSQVFLSIALPVSIIPLVMFTANKKIMGPFVNKKWVTITSSLVAIILTGLNIFLILQTLGWVQ

SEQ ID NO: 11 - protein, 455 aa, Lactobacillus brevis
MTDNVSAKSVQGDLTNGPSLAEINGSVRVPKEKGFVRNLLAFSGPGALVAVGYMDPGNWVTSIGGGAQYGYLLMSVILMSSLIAMLLQYMAAKLGIVTQMDLARATRAHTGKRIGAVLWVMTELAIMATDIAEVIGGAIALKLLFGVPLILGVSLTVLDVLLLLLLTRLGFRKIEAIVLCLILVILVVFAYEVVIAQPSMGQAVASFVPQAEIMRPGQLTMALGIVGATVMPHNLYLHSSIAQTRKFDRQDPAEMARAVKFTAWDSNIQLFGAFIINCLLLLLGAAMFFGKDAGALGTFGQLYDALQDNRLAGAVASPVLSTLFAVALLASGQNSTITGTLTGQVIMEGFINMRLPIWVRRLVTRLISVAPVIIVTILYGGSEQALDRLLVNSQVFLSIALPFSMIPLTIFTSSKRIMGTRWVNRWWVTALAWGCTAILTVLNIQIVWATMTTLF

SEQ ID NO: 12 - protein, 450 aa, Lactobacillus casei
MARPDERLTVQREKRSLDDINRSVQVPSVYESSFFQKFLAYSGPGALVAVGYMDPGNWLTALEGGSRYHYALLSVLLMSILVAMFMQTLAIKLGVVARLDLAQAIAAFIPHWSRICLWLINEAAMMATDMTGVVGTAIALKLLFGLPLMWGMLLTIADVLVVLLFLRFGIRRVELIVLVSILTVGIIFGIEVARADPSIGGIAGGFVPHTDILTNHGMLLLSLGIMGATIMPHNIYLHSSLAQSRKYDEHIPAQVTEALRFGKWDSNVHLVAAFLINALLLILGAALFYGVGGHVTAFQGVYNGLKNPMIVGGLASPLMSTLFAFALLITGLISSIASTLAGQIVMEGYLNIRMPLWERRLLTRLVTLIPIMVIGFMIGFSEHNFEQVIVYAQVSLSIALPFTLFPLVALTNRRDLMGIHVNSQLVRWVGYFLTGVITVLNIQLAISVFV

SEQ ID NO: 13 - protein, 451 aa, Lactobacillus rhamnosus
MTKRNEQLSVQQAKPSLDEINRSVQVPGVYEPSFVQKFLAYSGPGALVAVGYMDPGNWLTALEGGSRYHETLLAVLLLSILAAMFMQTLAIKLGVVARLDLAQAIAAFVPKWSRIGLWLVNEAAMMATDMTGVVGTAIALKLLFGLPLMWGMLLTIADVLVVLMFLRFGIRRIELIVLASILTVGIIFGIEVVRARPSMGGIVAGLVPHTEILTNRGMLLLSLGIMGATIMPHNIYLHSSLAQSRRYDEHIPAQVTEALRFGKWDSNVHLVAAFIINALLLILGATLFYGMSSHATAFEGVYNGLKNPAIVGGLASPLMSTLFAFALLITGLISSIASTLAGQIVMEGYLNIQIPLWARRLLTRLVTLIPIMIIGFVMGFSEQHFEQVIVYAQVALSIALPFTLFPLVALTDRRDLMGQHVNSPVVRWMGYVLTGIITLLNVQLILSVILP

SEQ ID NO: 14 - protein, 442 aa, Lactobacillus kefiri
MSQEPTHKSLDEINQSVEVPSVYETSFLQKFLAYSGPGALVAVGYMDPGNWLTSLSGGSQFRYALLSVLLMSILVAMFMQTLSIKLGVVARLDLAQAIAQKVPKSGRYTLWIINELAMMATDMTGVVGTAIALKLLFGLPLVYGILLTIFDVLLVLLFLRFGIRRIEFIVLAAILIVGVIFGIEVTRATPNIVEIAGGLIPTTHIVTNHEMLIMSLGIVGATIMPHNVYLHSSLAQSRRYDYHNPKQVNEALRFAKWDSNVHLVAAFLINALLLVLGGTLFFHTNSHFSAFQDVYNGLKSSAIVGSLASPLMSTLFAFALLITGMISSITSTLSGQIVMEGYLHIRLPLWERRLLTRFVTLIPILAIGFLVGFNDHDFEEIIVYAQIALSIALPFTLFPMVALTSNHDLMGVHTNRRYVTVIGYLLTSIITILNLQFVLASI

SEQ ID NO: 15 - protein, 439 aa, Lactobacillus alimentarius
MPNKKSLDEINESVKVPSVYDTSFLQKFLAYSGPGALVAVGYMDPGNWLTSLSGGSQYRYDLLSVLLISILVAMFMQTLSIKLGVVARLDLAQAIATKVSKPIRYFLWILNEIAMMATDLTGVIGTAIALKLLFNLPLVFGILLTVFDVLIVLIFLRFGIRRIEFIVLAAILTVGIIFGIEVFRAQPKLFSIISGVIPSTDLFTNHRKLVLSLGIVGATIMPHNIYLHSSLAQSRRYDHNDPLQVNEALRFAKWDSNVHLIAAFIINALLLVLGGTLFYHMTNQLASLQDVFTGLKSHAIVGTLASPLMSWLFAFALLITGMISSITSTLSGQIVMEGYLNIRLPLWQRRLLTRFVTLIPILIIGFIVHFNEQDFENLIVYAQIILSIALPFTLFPMIFLTNDKKIMGNHVNSKLTTTVGIILASAITILNLQLLFSLI

SEQ ID NO: 16 - protein, 441 aa, Lactobacillus plantarum
MQSHRHQSLEEINQSVAVPDVHQTAFWRKFLAYSGPGALVAVGYMDPGNWLTSLAGGGQFQYRLLAVLALAIIVAMFMQGLAIRLGVVARQDLAQAIASKLPRPVRYAAWILNEVAMMATDMTGVIGTAIALKMLFGLPLLAGILLTIADVLVVLLFLRFGIRRVEVIVLVAILTVGIIFGIEVGRAHVQFGNVLLGLVPTPLIVKNHTALVLSLGILGATIMPHNLYLHSSLAQSRRYDYHNPAQVTEALRFANWDSTVHLIAAFLINALLLVLGGTLFFGHTNALASLQAVFDGLKSTTVVGALASPVMSWLFALALLITGLISSITSTLAGQIVMEGYLHIRLPLWQRRLLTRAVTLIPILIIGMLVGFSDAAFENLIIYAQVALSIALPFTLLPLVALTNDASLMKAHVNRPAVTWVGYGLAGIITVLNIYLVYSLF

SEQ ID NO: 17 - protein, 434 aa, Lactobacillus reuteri
MERKSLDEINGSVDVPNVYQSAFWQKFLAYSGPGALVAVGYMDPGNWLTSLAGGSQYRYQLLVVLFTAILIAMYMQSLAIKLGVTTRTDLAQAIARRLPTPLRIALWLFNEIAMMATDLTGVVGTAVALNMLFKLPLLIGVLLTIADVLVVLFFLHFGIRRIEFIVLTAILVVGAIFAIEVCRAHPEFSAIMDGFVPRSTIFTNHSELLISLGIVGATIMPHNIYLHSSLAQSRRYDEHDPKQVKETLRFANWDSLIHLFAAFIVNALLLILGGTLFFHAASLGSLEDVFFGLKNPQIVGSLASPLMSWLFAFALLVTGLISSITSTLAGQIVMEGFINIRLPLWKRRLLTRAVTLVPILIIGFMINFKEEQFEQLIIYAQIVLSIALPFTLYPLVALTGNKKLMGPHVNSRWQTVLGYILASLVTGLNLLVLV

SEQ ID NO: 18 - protein, 440 aa, Lactobacillus crustorum
MTEKKSLDEINGSVAVPQYNTSFFRKFLAYSGPGALIAVGYMDPGNWLTSLVGGAHHKYQLLSVLLISILVATFMQSLSIRLGIASRQDLAQAIAKKAKKPVRYCLWIINELAMMATDLTGVIGTALALNMLFKLPLVFGVLITILDVFLILWFMRFGIRRIESIVVISILTVGLIFAFEVSHVQPNLTAIFKGFVPSQTIITNQNKLILSLGIIGATIMPHNIYLHSALAQSRRYDYHDSRQVREALRFANWDSIVHLIAALIINCLLLILGGTIFYDKADQLASLMTVFKGLMNYQVVGSLASSFMSYLFAFALLVTGLISSITSTLSGQIVMEGYLNIRLPLWQRRLLTRIITLIPILVIGFLVHFNEVIFEDLIVYAQIALSVALPFTLFPLVYLTNNAKIMGKHVNKKWQTILGFVLALIITILNIVLIATTLSH

SEQ ID NO: 19 - protein, 535 aa, Lactobacillus casei
MSDDHKKRHPIKLIQYANGPSLEEINGTVEVPHGKGFWRTLFAYSGPGALVAVGYMDPGNWSTSITGGQNFQYLLISVILMSSLIAMLLQYMAAKLGIVSQMDLAQAIRARTSKKLGIVLWILTELAIMATDIAEVIGAAIALYLLFHIPLVIAVLVTVLDVLVLLLLTKIGFRKIEAIVVALILVILLVFVYQVALSDPNMGALLKGFIPTGETFASSPSINGMSPIQGALGIIGATVMPHNLYLHSAISQTRKIDHKNPDDVAQAVKFSAWDSNIQLSFAFVVNCLLLVMGVAVFKSGAVKDPSFFGLFQALSDSSTLSNGVLIAVAKSGILSILFAVALLASGQNSTITGTLTGQVIMEGFVHMKMPLWARRLVTRIISVIPVIVCVMLTARDTPIQQHEALNTLMNNSQVFLAFALPFSMLPLLMFTNSKVEMGDRFKNTGWVKVLGWISVLGLTGLNLKGLPDSIAGFFGDHPTATQTNMANIIAIVLIVAILALLAWTIWDLYKGNQRYEAHLAAVADEKEAKADVDEQ

SEQ ID NO: 20 - protein, 535 aa, Lactobacillus rhamnosus
MSDDHKKKHSMKLIQYANGPSLEEINGTVEVPHGKGFWRTLFAYSGPGALVAVGYMDPGNWSTSITGGQNFQYLLISVILMSSLIAMLLQYMAAKLGIVSQMDLAQAIRARTSKKLGIVLWILTELAIMATDIAEVIGAAIALYLLFHIPLVIAVLVTVLDVLVLLLLTKIGFRKIEAIVVALILVILLVFVYQVALSDPNMGALLKGFIPTGETFASSPSVNGMSPIQGALGIIGATVMPHNLYLHSAISQTRKIDHKDPEDVAQAVKFSAWDSNIQLTFAFVVNCLLLVMGVAVFKSGAVKDPSFFGLFQALSDSSTLSNGVLIAVAKSGILSILFAVALLASGQNSTITGTLTGQVIMEGFIHMKMPLWARRLVTRVISVIPVIVCVMLTARETPIQQHEALNTLMNNSQVFLAFALPFSMLPLLMFTNSKVEMGDRFKNTGWVKVLGWVSVIGLTYLNLKGLPDSIAGFFGDNPTAAQTNIANMIAYVLIAAVLALLAWTIWDLYKGNKRYEAHLEAVADEEEAKANDDVQ

SEQ ID NO: 21 - protein, 530 aa, Lactobacillus plantarum
MSEKTNTPNRKHKLIEYANGPSLEEINGTIEVPKNLNFWKTLFAYSGPGALVAVGYMDPGNWSTSITGGQNYQYMLMSVILISSLIAMLLQYMAAKLGIVSQMDLAQAIRARTSKSLGIVLWILTELAIMATDIAEVIGAAIALYLLFNIPLVIAVFITVLDVLVLLLLTKIGFRKIEAIVVCLILVILFVFVYQVALSNPDWGGVIKGLVPTADTFSTSRSVNGMTPLSGALGIIGATVMPHNLYLHSAISQTRKIDHNDEEDVARTVKFAAWDSNIQLSFAFVVNSLLLIMGVAVFKSGAVKDPSFFGLYEALSNTSMLSNGILISVAKSGALSALFAIALLASGQNSTITGTLTGQVIMEGFVHMRMPLWLRRLVTRLISVIPVLICVLLTSGKSAIDEHTALNNLMNNSQVFLAFALPFSMLPLLMMTDSAAEMGKRFKNSLWIKGLGWLSVIGLTFLNLLGLPDSILGFFGDNPSAGEQTFSKILAYLLIAAILALLVWTVFDLQRGNKRYVEQQLAAAAKEANK

SEQ ID NO: 22 - protein, 530 aa, Pediococcus acidilactici
MSNEIKNPKKRRKLISYANGRSLEEINGTVKVPKNIGFWKTLFMYSGPGALVAVGYMDPGNWSTSITGGQNFQYMLMSIILISSLIAMLLQYMAAKLGIVSQMDLAQAIRARTSRALGIVLWILTELAIMATDIAEVIGAAIALYLLFHIPLVVAVFITVFDVLLLLLLTKIGFRKIEAIVVCLIMVILVVFVYQVALSHPSWGAVFGGLIPTTKAFATTPTVGGMTPLSGSLGIIGATVMPHNLYLHSAVSQTRKINHDDEEDVARTVRFSTWDSNIQLSFAFVVNALLLVMGVAVFKTGAVQDPSFFGLFHALNDTSTLSNGILIGVAKTGILSTLFAVALLASGQNSTITGTLTGQVIMEGFVHMRMPLWARRLITRLISVVPVLICVMLTSGKGTIQEHEALNNLMNNSQVFLAFALPFSMVPLLMMTDSRVEMGDRFKNSWIVRILGWISVIFLTYLNLTGLPDSIAAFFGENASAAEISMAHDIAYALIVAVLALLAWTVIELYKGNKRYEIELAEKANAKEAA

SEQ ID NO: 23 - protein, 521 aa, Lactobacillus salivarius
MVNNENNHKKHKMIQYANGKSLEEANGTVEIPKGKGFWKTLFAYSGPGALVAVGYMDPGNWSTSITGGQNFQYLLMSVILLSSLIAMLLQYMAAKLGIVSQMDLAQAIRARTSKALGIVLWILTELAIMATDIAEVIGAAIALYLLFDIPLIIAVFITVFDVLLLLLLTKVGFRKIEAIVVCLIFVILFVFVYQVALSNPDWGGVFKGLIPTSETFAKHPVVHDMSPLNGALGIIGATVMPHNLYLHSAISQTRKFDRNNEDDIANAVRFTAWDSNIQLGLAFVVNSLLLIMGVAVFKSGAVEDPSFFGLYQALSDTSVMSNGLLAAAARTGILSTLFAVALLASGQNSTITGTLTGQVIMEGFIHLRMPLWARRLITRLLSVIPVLICVALTSGKSTIEEHEALNNLMNNSQVFLAFALPFSMLPLVIMTGSKVEMGERFKNRLWINILGWISVISLTYLNMIGLPQNLEPFFPADKVGLAHTVAYILIVLIIALLIWTLVELHLGNKRFAAEQAKKHNK

SEQ ID NO: 24 - protein, 520 aa, Lactobacillus fermentum
MDNTKNQHRKLRLIEHANGKSLEEINGTVEVPHGKGFFRTLFAYSGPGALVAVGYMDPGNWSTSITGGQSFQYTLMTTILISSLIAMLLQYMAAKLGIVSQMDLAQAIRARTGKALGVILWLMTELAIMATDIAEVIGAAIALNLLFHIPLVLAVFITVLDVLVLLLLTKIGFRKIEAIVACLILVILAVFAYQVALSHPDWAGVFKGLLPTKEAIAKEPVVGGISPLTGSLGIIGATVMPHNLYLHSAISQTRKIDHTNAEDIKQTVRFTAWDSNIQLTLAFFVNALLLIMGVAVFKNGAVQDSSFFGLYDALNNTDMLSNGLLIAVAKSGVLSTLFAIALLASGQNSTITGTLTGQVIMEGFVHMKMPLWARRLITRLLSVVPVLVCVAMTAHESTIDQHASLNILMENSQVFLAFALPFSMLPLLIMTNSDTEMGQFKNSLWVRVLGWISVIGLTFLNLYNLPQTYEGFGIWSKGLSDVLAWISIVVIVVLLAWTCFELIRGDRRLAAEREKHTWEK

SEQ ID NO: 25 - protein, 533 aa, Lactobacillus amylolyticus
MCSRKVLLTKQKGKHYLIRYANGKSLSEINGTIEIPKKRTFWRMLWAYTGPGALVAVGYMDPGNWATSITGGQSFQYILMSTILISSLMAMLLQYMAAKLGIVTQMDLAQAIRLRTGKALGIVLWLMTELAIMATDIAEVIGAAIALNLLFDIPLVPAVFITVLDVLLLLLLARIGFRKIEAVVSCLILVILLVFVYEVLLSNPDWSKAFVGLVPSAKIIQTHPVVGGISPLTGTLGIIGATVMPHNLYLHSAISQTRKINHHNLQLIRDAVKYTALDSNIQLSLAFLVNALLLIMGAAVFKSGAVRDSSFFGLYQALDNAKMLSDPLLVHVARTGILSTLFAVALLASGQNSTITGTLTGQVIMEGYIHLKMPLWARRLVTRLLSVIPVLLCVSFTMNDSVMQQHFALNMLMENSQVFLAFALPFSVLPLLIMTNNKAEMGEFKNKPLWHYLGWACALVLTFLNLYNLPSQFVNFKFASKEVSTIIAYFVIVVIAALLLWTCIEIYIGDRKVKIHHSGFDAKEKELKEEGQK

(19) United States
(12) Patent Application Publication
(10) Pub. No.: US 2023/0292780 A1
(43) Pub. Date: Sep. 21, 2023

(54) LISTERIA INHIBITION BY MANGANESE DEPLETION

(71) Applicant: Chr. Hansen A/S, Hoersholm (DK)

(72) Inventors: Lieke VAN GIJTENBEEK, Hoersholm (DK); Solvej SIEDLER, Hoersholm (DK); Helle Skov GULDAGER, Hoersholm (DK); Louise Essendrup STEFFENSEN, Hoersholm (DK)

(73) Assignee: Chr. Hansen A/S, Hoersholm (DK)

(21) Appl. No.: 17/919,032

(22) PCT Filed: Apr. 16, 2021
(86) PCT No.: PCT/EP2021/059939

§ 371 Date: Oct. 14, 2022

(30) Foreign Application Priority Data
Apr. 16, 2020 (EP) 20169838.8
Aug. 18, 2020 (EP) 20191557.6

(51) Int. Cl. A23C 19/097; A23C 19/076; C12N 1/20

(52) U.S. Cl. CPC A23C 19/097; A23C 19/076; C12N 1/205; A23Y 2220/73; C12R 2001/225

(57) ABSTRACT

The present invention is in the field of food technology. The present invention provides methods of controlling growth of Listeria by limiting their access to manganese More specifically, the present invention provides a method of inhibiting or delaying growth of Listeria by reducing the manganese concentration in a food product which is preferably a dairy product. The invention also provides manganese scavengers and uses thereof to inhibit or delay Listeria growth.

### FIELD OF THE INVENTION

The present invention lies in the field of microbiology and relates to methods for controlling of Listeria growth. The invention also relates to food products and preparations thereof using the methods.

### BACKGROUND OF THE INVENTION

Bacterial contamination of food products is known to be responsible for the transmission of food borne illnesses. Listeriosis is a bacterial infection caused by Listeria monocytogenes and is a known cause for severe illness, including severe sepsis, meningitis, or encephalitis, sometimes resulting in lifelong harm and even death. In particular the elderly, unborn babies, newborns and immunocompromised persons are at risk of severe illness. In pregnant women listeriosis may cause stillbirth or spontaneous abortion, and preterm birth is common. Listeriosis may in less severe cases cause mild, self-limiting gastroenteritis and fever. As a consequence, a significant effort has been made to inhibit the growth of Listeria monocytogenes in food products.

It is known that Listeria tolerates refrigeration temperatures, relatively high concentrations of NaCl and anaerobic conditions in food products. It can grow in environments with a pH between 4.3 and 9.5. At a temperature of 30° C., some L. monocytogenes strains can grow down to a pH of 4.3. At refrigerated temperatures from 10-4° C., this value lies higher between 4.6 up to 5.2, respectively, below which growth is severely impeded.

Tolerance of L. monocytogenes to low pH is also linked to water activity (aw). It is commonly reported that the bacterium can grow at an aw higher than 0.92. Therefore, with respect to pH level, there are many foods that seem susceptible to the growth of L. monocytogenes.

In dairy products, milk heat treatment is not always sufficient to guarantee the absence of L. monocytogenes. It is known that a lack of hygiene or sanitation during the post-pasteurization or post-processing steps would also lead to contamination. Although incidences of listeriosis via cultured dairy products are typically linked to consumption of soft cheeses prepared from unpasteurized milk, outbreaks via consumption of soft cheeses prepared from pasteurized milk do occur. This is also true for cottage cheese, for which it has been shown that some vegetative L. monocytogenes cells present in raw milk can remain viable after pasteurization. However, the larger threat lies in a post-processing step typical for cottage cheese in which curd is mixed with a cream dressing. This creates a time span in which the product has an elevated pH and a temperature more amenable for growth of L. monocytogenes cells that either have survived pasteurization or were introduced as a fresh contamination during the mixing step. US guidelines therefore require that potassium sorbate or another protective additive has to be added to warm-filled cottage cheese to prevent contamination with L. monocytogenes.

Listeria contamination is especially relevant for ready-to-eat (RTE) foods that are not heat-treated and stored for prolonged periods of time at refrigerated temperatures. The European Commission (EC) has therefore established criteria to define the acceptability of a ready-to-eat (RTE) food, based on the presence/absence or enumeration of L. monocytogenes throughout the food supply chain for a given type of food. Since refrigeration alone is not enough to protect food products from listerial growth, extensive measures are taken to minimize chances of contamination. These include strict hygienic guidelines during food processing and the addition of preservatives that protect foods during the storage period. There is an increasing demand for natural, clean-label products. Chemical preservatives such as propionate, sorbate, benzoate, lactate, and acetate may be less desirable. The replacement of chemical preservatives with more natural solutions, like food-grade bacterial solutions in the form of live lactic acid bacteria (LAB), have therefore gained popularity.

It is known that Listeria growth can be inhibited by various bacteriocins, applied directly in purified or crude form. For example, nisin has been shown to be effective in the control of Listeria monocytogenes in dairy products. Pediocin (PA-1) was shown to reduce L. monocytogenes counts in cottage cheese, cream, and cheese sauce. So far, nisin is the only bacteriocin that has been officially employed in a purified format in the food industry and its use has been approved worldwide.

Bacteriocins can be applied to foods via a bacteriocin-producing lactic acid bacteria (LAB) as a part of fermentation process or starter culture. A number of applications of bacteriocin-producing LAB have been reported to control pathogens in milk, yogurt, and cheeses successfully.

However, bacteriocins are easily degraded due to their proteinaceous nature, which may result in a loss of antibacterial activity. Another drawback is the possible lack of compatibility between the bacteriocin-producing strain and the starter cultures required for fermentation. Therefore, such approach is limited in cases where the starter cultures are adversely affected by the bacteriocin or can actively degrade or hamper the bacteriocin production by the bacteriocin-producing strain. Further, a successful implementation of bacteriocin-producing culture may require the ability of the strain to produce bacteriocin under the manufacturing conditions for the product, including the availability of appropriate nutrients and fermentation conditions such as time and temperature.

Bacteriophages (or “phages”) can act as natural antimicrobials against food pathogens in the food industry. Bacteriophages infect specific bacteria and use the genomic material of the bacteria to produce new phages, ultimately destroying the bacterial cell. A number of commercial phage products have been approved by the U.S. Food and Drug Administration (FDA) that target L. monocytogenes in food products.

Some challenges are associated with bacteriophage-based anti-listerial strategy. The efficacy of a given bacteriophage depends on the specific phage receptors differing for various Listeria strains. Further, it has been observed that the titer of the bacteriophages are a deciding factor in the success of the application. However, manufacturing a high-titer product on a large scale often remains difficult. Another challenge in using bacteriophage is the emergence of phage-resistant strains. A broad host range or a cocktail of phages must be designed to target environmental L. monocytogenes.

To overcome one or more of the above mentioned disadvantages associated with current anti-listerial solutions, there is a need for an alternative strategy to control growth of Listeria in food products, particularly in products that have a pH and water activity aw prone to Listeria contamination.

### SUMMARY OF THE INVENTION

The inventors of the present invention have sought to find effective methods to manage Listeria growth and identified low manganese levels as an important growth constraint. In terms of trace elements, it has been known for decades that iron is important for the growth of Listeria monocytogenes and many studies have been made in this respect (Lechowicz, Justyna, and Agata Krawczyk-Balska. “An update on the transport and metabolism of iron in Listeria monocytogenes: the role of proteins involved in pathogenicity.” Biometals 28.4 (2015): 587-603.). However, it is shown for the first time the possibility to inhibit Listeria growth by limiting the manganese in the environment. The present invention is in part based on the surprising finding that by reducing the level of manganese in the food product, for example by removing manganese using manganese scavengers, the growth of Listeria can be reduced or delayed.

Manganese scavenging agents have been disclosed in WO2019/202003 to inhibit or delay fungal growth. As disclosed, a reduction in manganese concentration to 0.01 ppm was seen to have an effect. However, there is no suggestion that the strategy can be utilized against gram-positive pathogenic bacteria like Listeria. The inventors have shown that both Listeria innocua strains and Listeria monocytogenes strains are responsive to the methods described herein, and the concentration of manganese required for inhibition is lower than that required by yeast and mold.

The genus Listeria as of 2019 is known to contain 20 species: L. aquatica, L. booriae, L. cornellensis, L. costaricensis, L. goaensis, L. fleischmannii, L. floridensis, L. grandensis, L. grayi, L. innocua, L. ivanovii, L. marthii, L. monocytogenes, L. newyorkensis, L. riparia, L. rocourtiae, L. seeligeri, L. thailandensis, L. weihenstephanensis, and L. welshimeri two well-known species are Listeria monocytogenes or Listeria innocua. L. innocua and L. listeria have been found to behave similarly in dairy environment. Listeria innocua is generally considered nonpathogenic and is used as surrogate in pilot studies which reflect and predict inhibition of Listeria monocytogenes. In addition, a fatal case of Listeria innocua bacteremia has been reported (Perrin et al, Journal of Clinical Microbiology 41.11 (2003): 5308-5309).

Cases of human listeriosis are almost exclusively caused by the species L. monocytogenes. Listeria monocytogenes can be divided into 13 different serotypes all of which are able to cause listeriosis. However, most cases are caused by serotypes 1/2a, 1/2b and 4b.

The methods of the present invention can be used for manufacturing many type of dairy products, such as yoghurt or cheese. Cheeses are of particular focus because they are susceptible to Listeria contamination.

In preferred embodiments, the dairy product has a pH which is higher than 4.3 but lower than 7.0, such as higher than 4.4, such as higher than 4.5, such as higher than 4.6, such as higher than 4.7, such as higher than 4.8, such as higher than 4.9, such as higher than 5.0, such as higher than 5.1, such as higher than 5.2, such as higher than 5.3, such as higher than 5.4, such as higher than 5.5, such as higher than 5.6, such as higher than 5.7, such as higher than 5.8, such as higher than 5.9, such as higher than 6.0, such as higher than 6.1, such as higher than 6.2, such as higher than 6.3, such as higher than 6.4, such as higher than 6.5, such as higher than 6.6, such as higher than 6.7, such as higher than 6.8, such as higher than 6.9.

Manganese can be found naturally in many food sources including leafy vegetables, nuts, grains and animal products. Typical ranges of manganese concentrations in common foods are for example 0.04 to 0.1 ppm in cow milk, 0.4-40 ppm in grain products, 0.1-4 ppm in meat, poultry, fish and eggs, 0.4-7 ppm in vegetable products.

To combat the problem of microbial spoilage, the present invention provides in a first aspect a method of inhibiting or delaying growth of Listeria in a product comprising the step of reducing manganese present in said product. Manganese concentration can be reduced by the methods described in this invention. In a preferred embodiment, one or more manganese scavengers are added to reduce manganese. Manganese concentration is preferably reduced to below 0.006 ppm, such as below about 0.005 ppm, below about 0.004 ppm, below about 0.003 ppm, below about 0.002 ppm or below about 0.001 ppm. In preferred embodiments, the product is characterized by a manganese concentration of below 0.006 ppm, such as below about 0.005 ppm, below about 0.004 ppm, below about 0.003 ppm, below about 0.002 ppm, below about 0.001 ppm or lower. Using the method, a product in which growth of Listeria is hampered can be obtained. Such products are characterized by a low or lack of growth of Listeria when subject to a challenge test by artificially contaminating the product with Listeria. The method further comprises the step of measuring the manganese concentration in the product and obtaining a value of below 0.006 ppm.

In particular, the present invention provides a method of inhibiting or delaying Listeria growth in a food product, such as a ready-to-eat product, meat product, vegetable product or fermented food product prepared from milk such as yogurt or cheese. The method is characterized by the step of reducing manganese concentration in the food product in order to deprive the Listeria of manganese and thereby delaying or inhibiting their growth in the food product.

In one preferred embodiment, the present invention provides a method of inhibiting or delaying growth of Listeria monocytogenes in a food product comprising the step of reducing manganese present in said product.

In a second aspect, the present invention provides a method preparing a food product such as a fermented food product, comprising reducing manganese present in said food product. Manganese concentration can be reduced by the methods described in this invention or other methods known to a skilled person in the art. In a preferred embodiment, one or more manganese scavengers are added to reduce manganese. Manganese concentration is preferably reduced to below 0.006 ppm, such as below about 0.003 ppm or below about 0.001 ppm. Using the method, a food product comprising manganese concentration below 0.006 ppm can be obtained.

In a third aspect, the present invention provides food products such as ready-to-eat product, meat product or fermented food products obtained by the methods described herein. In one embodiment, the present invention provides a method of providing a food product, comprising the steps of reducing manganese in the product and obtaining the product, wherein the product comprises lactic acid bacteria as manganese scavenger.

In a further aspect, the present invention provides the use of one or more manganese scavengers to inhibit or delay Listeria growth as well as to produce food products. Manganese scavengers have the effect of making less manganese available in a product for Listeria, thus inhibiting or delaying their growth.

In another aspect, the present invention provides manganese scavengers, selections and uses thereof for Listeria inhibition.

### BRIEF DESCRIPTION OF THE FIGURES

FIG. 1a-e show growth curves of the four Listeria strains and L. lactis subsp. cremoris MG1363 in medium supplemented with increasing levels of manganese. Manganese was added in different concentrations: 0.6 ppm (black squares), 0.06 ppm (upright grey triangle), 0.006 ppm (upside down grey triangle), 0.0006 ppm (light grey diamond), 0.00006 ppm (light grey circle), 0 ppm (light grey square).

FIG. 2a shows the growth of L. innocua indicated by red fluorescence in model cottage cheese prepared with starter culture Lactococcus lactis subsp. lactis and Streptococcus thermophilus (“Fresco”), with starter culture supplemented with manganese (“Fresco +Mn”), with starter culture and manganese scavenging bacteria (“Fresco +32092”), or with starter culture and manganese scavenging bacteria, supplemented with manganese (“Fresco+32092+Mn”). FIG. 2b shows the acidification curves of model cottage cheese prepared with starter culture (“Fresco”), with starter culture supplemented with manganese (“Fresco+Mn”), with starter culture and manganese scavenging bacteria (“Fresco+32092”), or with starter culture and manganese scavenging bacteria, supplemented with manganese (“Fresco+32092+Mn”) (not inoculated with L. innocua).

FIG. 3 shows the CFU count of L. innocua in the model cottage cheese obtained at day 1, 8, 16 and 21.

FIG. 4 shows the absence of an L. innocua inhibition zone in a well diffusion assay performed using the supernatant of DSM 32092.

FIG. 5 shows the growth of L. lactis (FIG. 5a), S. thermophilus (FIG. 5b) and L. innocua (FIG. 5c) in chemically defined medium (CDM) supplemented with increasing levels of manganese: 0.6 ppm (black squares), 0.06 ppm (upright grey triangle), 0.006 ppm (upside down grey triangle), 0.0006 ppm (light grey diamond), 0.00006 ppm (light grey circle), 0 ppm (light grey square).

FIG. 6 shows the growth of L. monocytogenes in industrial cottage cheeses prepared with starter culture Lactococcus lactis subsp. lactis, Lactococcus lactis subsp. cremoris and Streptococcus thermophilus (“Fresco”), with starter culture supplemented with manganese (“Fresco+Mn”), with starter culture and manganese scavenging bacteria (“Fresco+32092”), or with starter culture and manganese scavenging bacteria, supplemented with manganese (“Fresco+32092+Mn”).

### DETAILED DESCRIPTION OF THE INVENTION

In response to the demand for a new strategy to control growth of Listeria in food products, the present invention provides a novel method of inhibiting or delaying Listeria growth in a product, in particular products having a pH above 4.3 and an aw higher than 0.92.

The method is based on the surprising finding that low manganese concentrations can serve as limiting factor for Listeria growth. Manganese is present in trace amounts in nature and many of our consumer goods. However, there has not yet been any report suggesting that by manipulating the concentration of manganese, Listeria growth can be effectively managed. Additionally, the inventors also discovered that lack of manganese did not restrain growth of Lactoccocus lactis subsp. cremoris, a gram-positive bacterium.

Based on this finding, it is envisioned that such strategy is applicable beyond food products for human consumption and extending to other food products such as animal feed and pet foods for animals.

The present invention provides in a first aspect a method of inhibiting or delaying Listeria growth in a product comprising depleting manganese in said product to a concentration of below 0.006 ppm.

In general, inhibiting means a decrease, whether partial or whole, in function and activity of cells or microorganisms. As used herein, the terms “to inhibit” and “inhibiting” in relation to Listeria mean that the growth, the number, or the concentration of Listeria is the same or reduced. This can be observed for example, by measuring the Listeria growth and comparing it with a control. Such control may be for example a product without manganese scavengers applied. Methods of determining Listeria growth inhibition or delay are known to a skilled person in the art.

The term “to delay” in general means the act of stopping, postponing, hindering, or causing something to occur more slowly than normal. To see whether there is a delay, one may compare the time needed for the Listeria to grow to a given level in two products, one of which with reduced manganese and the other one without (but otherwise the same). In some embodiments, “delaying growth of Listeria” refers to delaying by 7 days, such as 8, 9, 10, 11, 12, 13, 14, 15, 16, 17, 18, 20, 21, 22, 23, 24, 25, 30, 35, 40, 45, 50, 55, 60 days.

### LISTERIA DETECTION

The presence of Listeria can be determined using routine enumeration methods known in the art. One may apply standard protocols in US FDA's Bacteriological Analytical Manual (BAM) (Hitchins et al., “BAM: Detection and Enumeration of Listeria monocytogenes.” Bacteriological analytical manual (2016)) or protocols published by the European and International Standard method EN ISO 11290-1:2017 (ISO, PNEN. “11290-1: 2017. Microbiology of the food chain—Horizontal method for the detection and enumeration of Listeria monocytogenes and of Listeria spp.”). Other methods can also be used, such as described in Law et al. “An insight into the isolation, enumeration, and molecular detection of Listeria monocytogenes in food.” Frontiers in microbiology 6 (2015): 1227.

One advantage of the present invention is to ensure food safety by controlling growth of Listeria during the shelf life of dairy products. As used herein, the term “shelf life” means the period of time that a food product remains sellable to retail customers.

In some embodiments, food products prepared using the methods described in the present application may have a Listeria count of less than 100 cfu/g during the shelf life, for example, at day 1, 5, 10, 15, 20, 25, 30, 35, 40, 45, 50, 55 or 60 days, when stored at a temperature between 1-8° C.

In some embodiments, cheese products prepared using the methods described in the present application may have a Listeria count of less than 100 cfu/g at day 1, 2, 3, 4, 5, 6, 7, 8, 9, 10, 11, 12,13, 14, 15, 16, 17, 18, 19, 20, 21, 22, 23, 24, 25, 26, 27, 28, 29 or day 30 when stored at a temperature between 1-8° C.

The term “manganese” in accordance with the present invention refers to manganese which is present in a product (i.e. forming part of product, such as within the product or on the surface of a product) that is available to be taken up by Listeria. Availability as used herein refers to the ability of Listeria to transport into the cell. Such availability is known or can be readily determined by a skilled person in the art. For instance, manganese cannot be taken up if it is bound in chelated form.

In one preferred embodiment, the present invention is directed to a method of inhibiting or delaying growth of Listeria in a food product, comprising reducing manganese concentration in a food matrix of the food product. As used herein, the term “food matrix” refers to the food's composition and structure.

The term “reduce” or “reducing” generally means lowering the amount of a substance in a given context. As used herein, the term “to reduce manganese” or “reducing manganese” means to reduce the amount of manganese present in a food product that is available to be taken up by Listeria. This can be achieved by making the it unavailable for Listeria uptake using the methods disclosed herein.

For example, this can be carried out by removing manganese present in the food product or in a material which is to become part of the product. For example, depending on the material, this can be carried out by subjecting the raw material ion exchange chromatography to remove manganese so that the concentration in the final product is reduced.

Given that Listeria may first come into contact with a product on the surface, it is within the spirit of the present invention that the step of reducing is carried out on parts of the product, for example in the exterior part of the product such as the coating, packaging or an outer layer. This can be achieved in some embodiments by spraying or applying a composition according to the present invention to the exterior of food product.

Manganese concentration or manganese level as used herein is expressed in parts per million (“ppm”) calculated on a weight/weight basis. Reducing manganese in a product to a concentration below a value means reducing manganese in the product or parts thereof such that the concentration of manganese in the entire product by weight is reduced. Methods of determining trace elements such as manganese are known in the field of food analysis.

In applying the present methods, one skilled in the art may first determine the manganese level which is present in the products to be treated. Manganese concentration for food products is well studied and can be found in national food composition databases such as Danish Food Composition Databank and Canadian Nutrient Files. In general, manganese is present at a concentration of at least 0.03 ppm for milk, making dairy products susceptible for Listeria contamination. Manganese levels have been reported to range from 0.04 to 0.1 ppm in cow milk and up to 0.18 ppm in goat or sheep milk (Muehlhoff et al., Milk and dairy products in human nutrition. Food and Agriculture Organization of the United Nations (FAO), 2013). As for fermented milk products like cheese, the manganese level usually increases due to the concentration process from milk, often up to 10-fold or more. Different levels have been reported for various types of cheeses, for example about 0.06 ppm for ricotta cheese, 0.11 ppm for cream cheese, 0.34 ppm for brie, 0.3 ppm for mozzarella, 0.7 ppm for cottage cheese, 0.68 ppm for gouda and 0.74 ppm for cheddar cheese (Smit, L. E., et al. The nutritional content of South African cheeses. ARC-Animal Improvement Institute, 1998; Gebhardt, Susan, et al. “USDA national nutrient database for standard reference, release 12.” United States Department of Agriculture, Agricultural Research Service, 1998).

Manganese in a product is preferably reduced to a concentration below 0.006 ppm, below about 0.005 ppm, below about 0.004 ppm, below about 0.003 ppm, below about 0.002 ppm, below about 0.001 ppm, below about 0.0009 ppm, below about 0.0008 ppm, below about 0.0007 ppm, below about 0.0006 ppm, below about 0.0005 ppm, below about 0.0004 ppm, below about 0.0003 ppm, below about 0.0002 ppm, below about 0.0001 ppm or lower.

As used herein, the term “about” indicates that values slightly outside the cited values, i.e., plus or minus 0.1% to 10%. Thus, concentrations slightly outside the cited ranges are also encompassed by the scope of the present inventions.

In one embodiment, the present invention provides a method of inhibiting or delaying growth of Listeria in a product, preferably a food product, comprising the steps of
reducing manganese in the product, and
obtaining the product where manganese concentration is below 0.006 ppm in the product.

The present method further comprises the step of measuring the concentration of manganese. This can be performed after the reducing step so to determine whether the concentration of manganese is reduced. In one embodiment, the present invention provides a method of inhibiting of delaying growth of Listeria in a food product, comprising reducing manganese in the product to a concentration of below 0.006 ppm in the product, and measuring the manganese in the product, and optionally obtaining a value of below 0.006 ppm.

In one embodiment, the present invention provides a method of inhibiting or delaying growth of Listeria in a product, comprising the steps of
reducing manganese in the product to a concentration of below 0.006 ppm in the product,
measuring the concentration of the manganese in the product and obtaining a value of below 0.006 ppm.

Methods of measuring of manganese at low concentration are well known to a person skilled in the art. Such methods include atomic absorption spectroscopy, atomic emission spectroscopy, mass spectrometry, neutron activation analysis and x-ray fluorimetry (see e.g., Williams et al. “Toxicological profile for manganese.” (2012)).

Manganese concentration can be measured according the standard procedure as described in “Foodstuffs—Determination of trace elements—Pressure digestion” in European Standard EN13805:2014 published by European Committee for Standardization or as described in “Water quality—Determination of selected elements by inductively coupled plasma optical emission spectrometry (ICP-OES)” in ISO 11885:2007 published by International Organization for Standardization.

It is also possible to subject the product to a model microorganism, such as Listeria or other bacteria, whose growth can be used to indicate the level of manganese in the product.

### Removal of Manganese

Methods of removing manganese are known in the art. Manganese is a common contaminant in many mine waters, groundwater, and freshwaters. In waste water treatment, manganese ions can be chemically removed from effluents by oxidation to MnO₂, adsorption, or precipitation as a carbonate.

Alternatively, manganese removal can involve biological processes as alternatives to chemical routes. The role of microbial activity in the remediation of manganese-contaminated waters has been described in various literatures, e.g. Burger et al. Manganese removal during bench-scale biofiltration. Water Research. 2008;42(19):4733-4742; Johnson et al. Rapid manganese removal from mine waters using an aerated packed-bed bioreactor. Journal of Environmental Quality. 2005;34(3):987-993; Tekerlekopoulou et al. “Removal of ammonium, iron and manganese from potable water in biofiltration units: a review.” Journal of Chemical Technology and Biotechnology 88.5 (2013): 751-773; Patil et al. “A review of technologies for manganese removal from wastewaters.” Journal of Environmental Chemical Engineering 4.1 (2016): 468-487. In one embodiment, the step of reducing manganese in the product comprises using ion-exchange chromatography. This is especially applicable if the product is in liquid or substantially liquid.

In one preferred embodiment, the step of reducing manganese in the product is carried out by adding a manganese scavenger. As used herein, the term “manganese scavenger” refers to a material that is capable of making manganese unavailable for Listeria. The material can be a chemical material, such as a chemical chelating material selected from the group consisting of ethylenediaminetetraacetic acid (EDTA), ethylene glycol-bis(β-aminoethyl ether)-N,N,N′,N′-tetraacetic acid (EGTA), diaminocyclohexanetetraacetic acid (DCTA), nitrilotriacetic acid (NTA), 1,2-bis(o-aminophenoxy)ethane-N,N,N′,N′-tetraacetic acid (BAPTA) or diethylenetriaminepentaacetic acid (DTPA), preferably the chemical chelating material is ethylenediaminetetraacetic acid (EDTA). The material can also be a biological material, such as bacteria.

In some embodiments, the chemical chelating material is hydrocolloids, preferably food hydrocolloids. Hydrocolloids are colloidal substances with an affinity for water. They may be isolated from plants, obtained by fermentation or plant-derived. Some hydrocolloids like galactomannans or natural gums are able to form complexes with metals and are therefore suitable to be used for the purpose of the present invention.

In some preferred embodiment the manganese scavenger is one or more bacteria strains, preferably a lactic acid bacteria strain, more preferably of the family of Lactobacillaceae and most preferably of the genus Lactobacillus. In such cases, it should be noted that “manganese” in the context of the present application does not include the manganese which is found intracellularly, as it is not available for Listeria uptake. Rather, manganese refers to the manganese that is found extracellularly and not bound by other substances for example in chelated form, i.e. in the cell-free parts of the product, since they would be available to be taken up by Listeria. Thus, in such cases, concentration of manganese should be measured taking only extracellular manganese into account. This can be done for example by removing cells (such as starter cultures) by centrifugation and obtaining cell-free supernatant, followed by measuring the manganese in the cell-free supernatant. As used herein, the term “bacteria strain” has its common meaning in the field of microbiology and refers to a genetic variant of a bacterium.

In one embodiment, the manganese scavenger is one or more bacteria strains which produce bacteriocin under conditions that are inducive for bacteriocin production. It is known in the art that production of bacteriocins by LAB generally depends on bacterial growth, and the maximum activity is usually coincident with maximum cell growth (Trinetta, Valentina, Manuela Rollini, and Matilde Manzoni. “Development of a low cost culture medium for sakacin A production by L. sakei.” Process Biochemistry 43.11 (2008): 1275-1280). A skilled person in the art is able to determine whether a given LAB would be able to produce bacteriocin. This can be done for example by examining the genome or by culturing the cell in suitable conditions and detect the presence of bacteriocin. In a more preferred embodiment, the manganese scavenger is one or more bacteria strains which do not produce bacteriocin under conditions that generally known to induce bacteriocin production.

In one embodiment, the present invention provides a method of inhibiting or delaying growth of Listeria in a product, comprising the steps of
selecting one or more bacteria strains as a manganese scavenger, and
reducing manganese in the product, preferably to a concentration of below 0.006 ppm in the product by adding the manganese scavenger.

According to preferred embodiments of the present invention, the method comprises selecting a bacteria strain having manganese uptake activities as a manganese scavenger. The selection is based on whether the bacteria strain has manganese transport systems.

Manganese scavenging bacteria can also be selected by providing a bacterium and confirming whether the bacteria would be able to inhibit yeast such as D. hansenii via challenge test, and if so, whether the inhibition is abolished by addition of manganese. Such methods can be routinely applied and are described in WO2019/202003 and by Siedler et al. “Competitive exclusion is a major bioprotective mechanism of lactobacilli against fungal spoilage in fermented milk products.” Applied and environmental microbiology 86.7 (2020). High throughput method may be advantageously applied to select suitable bacteria from a pool of bacteria.

Manganese is involved in many crucial biological processes and is ubiquitously found in all organisms. Manganese also contributes to protection against oxidative stress and can also contribute to the catalytic detoxification of reactive oxygen species. Many bacteria have developed sophisticated acquisition system to scavenge essential metals from the environment, using low and high affinity transport systems for chelated or free metals. Manganese which is taken up by bacteria forms a large complex of nondialyzable polyphosphate-protein aggregates in the protein which may reach very high intracellular concentrations.

Transport systems for manganese have been studied and are for example described in Kehres et al., “Emerging themes in manganese transport, biochemistry and pathogenesis in bacteria.” FEMS microbiology reviews 27.2-3 (2003): 263-290.

In one embodiment, a bacteria strain having manganese uptake activities comprises bacterial Mn²⁺ transporters. Mn²⁺ transporters may be an ABC transporter (for example SitABCD and YfeABCD) or a proton-dependent Nramp-related transport system belonging to the family designated as TC #3.A.1.15 and TC #2.A.55 in the transporter classification system given by the Transport Classification Database (M. Saier; U of CA, San Diego, Saier M H, Reddy V S, Tamang D G, Vastermark A. (2014)). The TC system is a classification system for transport proteins which is analogous to the Enzyme Commission (EC) system for classification of enzymes. The transporter classification (TC) system is an approved system of nomenclature for transport protein classification by the International Union of Biochemistry and Molecular Biology. TCDB is freely accessible at http://www.tcdb.org which provides several different methods for accessing the data, including step-by-step access to hierarchical classification, direct search by sequence or TC number and full-text searching.

In one embodiment, the method comprises selecting a bacteria strain comprising a protein belong to the family designated as TC #3.A.1.15 (manganese chelate uptake transporter (MZT) family) as manganese scavenger.

For example, the manganese scavenger is a bacteria strain comprising a manganese chelate uptake transporter designated as TC #3.A.1.15.2, TC #3.A.1.15.6, TC #3.A.1.15.8, TC #3.A.1.15.14 or functional variants thereof.

While the ABC transporter is mainly active at higher pH, proton driven transporters may be more active under acidic conditions. This makes them particularly useful as manganese scavengers in fermented food products. Thus, in one embodiment, a bacteria strain comprising a protein belong to the family designated as TC#2.A.55 (the metal ion (Mn²⁺-iron) transporter (Nramp) family) is selected.

The step of selecting one or more bacteria strains as manganese scavenger comprises determining whether one or more bacteria strain comprise a manganese transporter designated as TC #2.A.55 or functional variants thereof.

More preferably, the transporter belongs to the subfamily designated as TC #2.A.55.2 or the subfamily designated as TC #2.A.55.3, as manganese scavenger.

For example, the manganese scavenger is a bacteria strain comprising a metal ion (Mn²⁺-iron) transporter (Nramp) designated as TC #2.A.55.3.1, TC #2.A.55.3.2, TC #2.A.55.3.2, TC #2.A.55.3.3, TC #2.A.55.3.4, TC #2.A.55.3.5, TC #2.A.55.3.6,

TC #2.A.55.3.7, TC #2.A.55.3.8 or TC #2.A.55.3.9 or functional variants thereof as manganese scavenger.

Most preferably, the method comprises selecting a bacteria strain comprising a protein designated as TC #2.A.55.2.6 or functional variants thereof as manganese scavenger.

In one embodiment, the manganese scavenger is a lactic acid bacterium. Preferably, the manganese scavenger is a bacteria strain of the family of Lactobacillaceae or of the genus Lactobacillus.

Preferably, the manganese scavenger is selected from the group consisting of Lactobacillus plantarum, Lactobacillus fermentum, Lactobacillus reuteri, Lactobacillus sakei, Lactobacillus brevis, Lactobacillus casei, Lactobacillus paracasei, Lactobacillus salivarius, Lactobacillus alimentarius, Pediococcus acidilactici, Lactobacillus rhamnosus and Lactobacillus kefiri.

The term “functional variant” is a protein variant having a substantially similar biological activity, i.e. manganese uptake activities.

As used herein, a “variant” refers to a variant form of a protein which shares at least 55%, 60%, 65%, 70%, 75%, 80%, 85%, 90%, 95%, 96%, 97%, 98%, 99% or 100% sequence identity with a particular nucleic acid or amino acid sequence of the protein.

The invention additionally provides polypeptide sequences of manganese transporters for selecting suitable manganese scavengers to carry out the present invention.

In one preferred embodiment, a manganese scavenger is a bacteria strain comprising a polypeptide having the sequence of SEQ ID NO: 1 (MASEDKKSKREHIIHFEDTPSKSLDEVNGSVEVPHNAGFWKTLAAYTGPGILVAVGYMDPGNWI TSIAGGASFKYSLLSVILISSLIAMLLQAMAARLGIVTGRDLAQMTRDHTSKAMGGFLWVITELAI MATDIAEIIGSAIALKLLFNMPLIVGIIITTADVLILLLLMRLGFRKIEAVVATLVLVILLVFAYEVILAQ PNVPELLKGYLPHADIVTNKSMLYLSLGIVGATVMPHDLFLGSSISQTRKIDRTKHEEVKKAIKFST IDSNLQLTMAFIVNSLLLILGAALFFGTSSSVGRFVDLFNALSNSQIVGAIASPMLSMLFAVALLAS GQSSTITGTLAGQIIMEGFIHLKMPLWAQRLLTRLMSVTPVLIFAIYYHGNEAKIENLLTFSQVFLSI ALPFAVIPLVLYTSDKKIMGEFANRAWVKWTAWFISGVLIILNLYLIAQTLGFVK) or functional variants thereof.

In other preferred embodiments, a manganese scavenger is a bacteria strain comprising a polypeptide having at least 55%, such as at least 60%, 65%, 70%, 75%, 80%, 85%, 90%, 95%, 96%, 97%, 98%, 99% or 100% sequence identity with the sequence of SEQ ID NO: 1.

Table 1 shows exemplary sequences which encodes functional variants of SEQ ID NO: 1 and their sequence identity with SEQ ID NO: 1.

TABLE 1

| | SEQ | Protein | % |
|---|---|---|---|
| Origin | ID NO | ID | identity |
| Lactobacillus casei | 4 | WP_013245308.1 | 99.8 |
| Lactobacillus brevis | 5 | WP_011667125.1 | 76.5 |
| Pedicoccus acidilactici | 6 | WP_070366435.1 | 76.4 |
| Lactobacillus plantarum | 7 | YP_004888316.1 | 74.3 |
| Lactobacillus sakei | 8 | WP_089535161.1 | 67.9 |
| Lactobacillus alimentarius | 9 | WP_057737113.1 | 68.3 |
| Lactobacillus floricola | 10 | WP_056974015.1 | 65.1 |
| Lactobacillus brevis | 11 | WP_011667396.1 | 57.6 |

In one preferred embodiment, a manganese scavenger is a bacteria strain comprising a polypeptide having the sequence of SEQ ID NO: 2 (MARPDERLTVQREKRSLDDINRSVQVPSVYESSFFQKFLAYSGPGALVAVGYMDPGNWLTALEG GSRYHYALLSVLLMSILVAMFMQTLAIKLGVVARLDLAQAIAAFIPNWSRICLWLINEAAMMATDM TGVVGTAIALKLLFGLPLMWGMLLTIADVLVVLLFLRFGIRRIELIVLVSILTVGIIFGIEVARADPSI GGIAGGFVPHTDILTNHGMLLLSLGIMGATIM PHNIYLHSSLAQSRKYDEHIPAQVTEALRFGKW DSNVHLVAAFLINALLLILGAALFYGVGGHVTAFQGAYNGLKNPMIVGGLASPLMSTLFAFALLITG LISSIASTLAGQIVMEGYLNIRM PLWERRLLTRLVTLIPIMVIGFMIGFSEHNFEQVIVYAQVSLSIA LPFTLFPLVALTN RRDLMGIHVNSQLVRWVGYFLTGVITVLNIQLAISVFV) or functional variants thereof.

In other preferred embodiments, a manganese scavenger is a bacteria strain comprising a polypeptide having at least 55%, such as at least 60%, 65%, 70%, 75%, 80%, 85%, 90%, 95%, 96%, 97%, 98%, 99% or 100% sequence identity with the sequence of SEQ ID NO: 2.

Table 2 shows exemplary sequences which encode functional variants of SEQ ID NO: 2 and their sequence identity with SEQ ID NO: 2.

TABLE 2

| | SEQ | Protein | % |
|---|---|---|---|
| Origin | ID NO | ID | identity |
| Lactobacillus casei | 12 | WP_012491767.1 | 99.3 |
| Lactobacillus rhamnosus | 13 | WP_005712842.1 | 85.6 |
| Lactobacillus kefiri | 14 | WP_054768793.1 | 72.4 |
| Lactobacillus alimentarius | 15 | WP_057738992.1 | 68.7 |
| Lactobacillus plantarum | 16 | YP_004889177.1 | 67.4 |
| Lactobacillus reuteri | 17 | WP_003668901.1 | 65.8 |
| Lactobacillus crustorum | 18 | WP_075887252.1 | 61.4 |

In one preferred embodiment, a manganese scavenger is a bacteria strain comprising a polypeptide having the sequence of SEQ ID NO: 3 (MSDDHKKRHPIKLIQYANGPSLEEINGTVEVPHGKGFWRTLFAYSGPGALVAVGYMDPGNWST SITGGQNFQYLLISVILMSSLIAM LLQYMAAKLGIVSQM DLAQAIRARTSKKLGIVLWILTELAIMA TDIAEVIGAAIALYLLFHIPLVIAVLVTVLDVLVLLLLTKIGFRKIEAIVVALILVILLVFVYQVALSDPN MGALLKGFIPTGETFASSPSINGMSPIQGALGIIGATVMPHNLYLHSAISQTRKIDYKNPDDVAQA VKFSAWDSNIQLSFAFVVNCLLLVMGVAVFKSGAVKDPSFFGLFQALSDSSTLSNGVLIAVAKSG ILSILFAVALLASGQNSTITGTLTGQVIMEGFVHMKMPLWARRLVTRIISVIPVIVCVMLTARDTPI QQHEALNTLMNNSQVFLAFALPFSMLPLLMFTNSKVEMGDRFKNTGWVKVLGWISVLGLTGLNL KGLPDSIAGFFGDHPTATQTNMANIIAIVLIVAILALLAWTIWDLYKGNQRYEAHLAAVADEKEAK ADVDEQ) or functional variants thereof.

In other preferred embodiments, a manganese scavenger is a bacteria strain comprising a polypeptide having at least 55%, such as at least 60%, 65%, 70%, 75%, 80%, 85%, 90%, 95%, 96%, 97%, 98%, 99% or 100% sequence identity with the sequence of SEQ ID NO: 3.

Table 3 shows exemplary sequences which encode functional variants of SEQ ID NO: 3 and their sequence identity with SEQ ID NO: 3.

TABLE 3

| | SEQ | Protein | % |
|---|---|---|---|
| Origin | ID NO | ID | identity |
| Lactobacillus casei | 19 | WP_003567390.1 | 99.8 |
| Lactobacillus rhamnosus | 20 | WP_005686822.1 | 95 |
| Lactobacillus plantarum | 21 | YP_004890566.1 | 76.3 |
| Pediococcus acidilactici | 22 | WP_065124048.1 | 76.2 |
| Lactobacillus salivarius | 23 | YP_535797.1 | 72.7 |
| Lactobacillus fermentum | 24 | WP_012391805.1 | 69.6 |
| Lactobacillus amylolyticus | 25 | WP_080881380.1 | 63.7 |

For purposes of the present invention, the degree of “sequence identity” between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al.ᵣ 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

In one embodiment, the selecting step comprises determining whether the bacteria strain comprises a manganese transporter having at least 55%, such as at least 60%, 65%, 70%, 75%, 80%, 85%, 90%, 95%, 96%, 97%, 98%, 99% or 100% sequence identity with the sequences of any one of SEQ ID NO: 1-3. The determination can be based on sequencing the bacteria strain or a blast search in known sequence databases.

The manganese scavengers used in the Examples sections in the present invention have manganese transporter as encoded SEQ ID NO: 1-3 or functional variants thereof.

In other embodiments, the present invention provides a method of inhibiting or delaying growth of Listeria in a product, comprising the steps of:
selecting one or more bacteria strains as manganese scavenger, and
reducing manganese in the product preferably to a concentration of below 0.006 ppm in the product by adding the manganese scavenger,
   wherein the selecting step comprises measuring a manganese uptake activity of one or more bacteria strains.

Manganese uptake activities can be measured using routine methods known in the art, see e.g. Kehres et al. “The NRAMP proteins of Salmonella typhimurium and Escherichia coli are selective manganese transporters involved in the response to reactive oxygen.” Molecular microbiology 36.5 (2000): 1085-1100.

For fermented food products such as fermented milk products, a manganese scavenger is preferably a Lactobacillus species. Different manganese transporter families are present in Lactobacillus and oftentimes multiple homologs of these are present as well. WO2019/202003 provides an overview of the phylogeny of the manganese transporter MntH family within Lactobacillus species (FIG. 11). As shown, manganese transporters can be found across the Lactobacillus species.

In preferred embodiments, the manganese scavenger is a bacteria strain selected from the group consisting of L. rhamnosus, L. salivarius, L. casei, L. paracasei, L. fermentum, L. sakei, L. reuteri, L. plantarum, L. brevis, L. kefiri, L alimentarius and Pedicoccus acidilactici. On the other hand, such transporter appears to be absent in L. helveticus, L. acidophilus, L. gasseri, and L. delbrueckii subsp. bulgaricus, making them less suitable for removing manganese.

According to a preferred embodiment of the present invention, the method comprises a selecting step of determining that the one or more bacteria strain(s) are free of a superoxide dismutase, preferably free of a manganese superoxide dismutase.

Superoxide dismutases, such as manganese superoxide dismutase, have been studied and are for example described in Kehres et al., “Emerging themes in manganese transport, biochemistry and pathogenesis in bacteria.” FEMS microbiology reviews 27.2-3 (2003): 263-290; Culotta V.0 “Superoxide dismutase, oxidative stress, and cell metabolism” Curr. Top. Cell Regul. 36, 117-132 (2000) or Whittaker 3.W “Manganese superoxide dismutase” Met. Ions Biol. Syst. 37, 587-611 (2000), Sanders et al. “Stress response in Lactococcus lactis: cloning, expression analysis, and mutation of the lactococcal superoxide dismutase gene.” Journal of Bacteriology 177.18 (1995): 5254-5260. among others. Superoxide dismutase catalyzes the conversion of superoxide radical to hydrogen peroxide has the enzyme commission number EC 1.15.1.1.

In the context of the present invention, the term “free of” means that genome of the one or more bacteria strains do not present a gene coding for a superoxide dismutase, or even if the genome of the one or more bacteria strains present a gene coding for a superoxide dismutase, this gene is not express by the one or more bacteria strains to produce superoxide dismutase with activity. Determination of whether a given bacterium is free of superoxide dismutase can be done by routine methods in the art, for example, by checking the presence of genes which codes for superoxide dismutase. Determination of superoxide dismutase activity can for example be done according to the method described in Beauchamp and Fridovich. “Superoxide dismutase: improved assays and an assay applicable to acrylamide gels.” Analytical biochemistry 44.1 (1971): 276-287 or in Misra and Fridovich. “Superoxide dismutase and peroxidase: a positive activity stain applicable to polyacrylamide gel electropherograms.” Archives of biochemistry and biophysics 183.2 (1977): 511-515.

It is however also possible that manganese scavenging bacteria can comprise both superoxide dismutase and a metal ion (Mn²⁺-iron) transporter (Nramp), as for example, but not limited to, Lactobacillus paracasei DSM 25612.

In some embodiments, the present application provides a method of inhibiting or delaying growth of Listeria in a food product, comprising the steps of selecting one or more bacteria strains and/or a chemical chelating material as the manganese scavenger, and adding one or more manganese scavengers, preferably as a Direct Vat Set (DVS) culture composition, to reduce manganese in the food product. Preferably, the bacteria strains comprise a manganese transporter designated as TC #2.A.55 or functional variants thereof.

In some embodiments, the present application provides a method of inhibiting or delaying growth of Listeria in a food product, comprising the steps of selecting one or more bacteria strains as the manganese scavenger, and adding one or more manganese scavengers as a Direct Vat Set (DVS) culture composition, preferably frozen or freeze-dried, to reduce manganese in the food product such as fermented food product like fermented dairy products. Preferably, the bacteria strains comprise a manganese transporter designated as TC #2.A.55 or functional variants thereof.

In one embodiment, the present application provides a method of inhibiting or delaying growth of Listeria in a fermented food product, such as fermented dairy products like cheese, comprising the steps of selecting one or more bacteria strains of the Lactobacillaceae family, preferably of the Lactobacillus strain, as the manganese scavenger, and adding one or more manganese scavengers as a Direct Vat Set (DVS) culture composition, preferably frozen or freeze-dried, to reduce manganese in the food product. Preferably, the selected bacteria strain comprises a manganese transporter designated as TC #2.A.55 or functional variants thereof and, optionally, does not produce bacteriocin.

In some embodiments, the present application provides a method of inhibiting or delaying growth of Listeria in a fermented food product preferably having a pH higher than 4.3 and/or a water activity of higher than 0.92, such as fermented dairy products like cheese, comprising the steps of selecting one or more bacteria strains of the Lactobacillaceae family, preferably of the Lactobacillus strain, as the manganese scavenger, and adding one or more manganese scavengers as a Direct Vat Set (DVS) culture composition, preferably frozen or freeze-dried, to reduce manganese in the food product. Preferably, the selected bacteria strain comprises a manganese transporter designated as TC #2.A.55 or functional variants thereof and, optionally, does not produce bacteriocin.

The selecting steps may comprise determining whether the one or more bacteria strains comprise a manganese transporter having at least 55%, such as at least 60%, 65%, 70%, 75%, 80%, 85%, 90%, 95%, 96%, 97%, 98%, 99% or 100% sequence identity with the sequence of any one of SEQ ID NO: 1-3. The selecting step may also comprise determining that the one or more bacteria strains are free of a superoxide dismutase, preferably free of a manganese superoxide dismutase, and, optionally, measuring a manganese uptake activity of the one or more bacteria strains.

In some embodiments, the present application provides a method of inhibiting or delaying growth of Listeria in a food product, such as fermented dairy products like cheese, comprising the steps of selecting one or more bacteria strains of the Lactobacillaceae family, preferably Lactobacillus plantarum, Lactobacillus fermentum, Lactobacillus reuteri, Lactobacillus sakei, Lactobacillus brevis, Lactobacillus casei, Lactobacillus paracasei, Lactobacillus salivarius, Lactobacillus alimentarius, Pediococcus acidilactici Lactobacillus rhamnosus and Lactobacillus kefiri, as the manganese scavenger, and adding one or more manganese scavengers as a Direct Vat Set (DVS) culture composition, preferably frozen or freeze-dried, to reduce manganese in the food product. Preferably, the bacteria strain comprises a manganese transporter designated as TC #2.A.55 or functional variants thereof and, optionally, does not produce bacteriocin.

In some embodiments, the present application provides a method of inhibiting or delaying growth of Listeria in a fermented dairy products like cheese, comprising the steps of selecting one or more bacteria strains of the Lactobacillus genus, preferably Lactobacillus plantarum, Lactobacillus fermenturn, Lactobacillus reuteri, Lactobacillus sakei, Lactobacillus brevis, Lactobacillus casei, Lactobacillus paracasei, Lactobacillus salivarius, Lactobacillus alimentarius, Lactobacillus rhamnosus and Lactobacillus kefiri, as the manganese scavenger, and adding one or more manganese scavengers as a Direct Vat Set (DVS) culture composition, preferably frozen or freeze-dried, to reduce manganese in the food product.

In some embodiments, the present application provides a method of inhibiting or delaying growth of Listeria in a fermented dairy products like cheese, comprising the steps of selecting two bacteria strains of the Lactobacillus genus, preferably selected from the group consisting of Lactobacillus plantarum, Lactobacillus fermentum, Lactobacillus reuteri, Lactobacillus sakei, Lactobacillus brevis, Lactobacillus casei, Lactobacillus paracasei, Lactobacillus salivarius, Lactobacillus alimentarius, Lactobacillus rhamnosus and Lactobacillus kefiri, as the manganese scavenger, and adding one or more manganese scavengers as a Direct Vat Set (DVS) culture composition, preferably frozen or freeze-dried, to reduce manganese in the food product. Preferably, the manganese in the product is reduced to a concentration of below 0.006 ppm, preferably below about 0.005 ppm, below about 0.004 ppm, below about 0.003 ppm, below about 0.002 ppm or below about 0.001 ppm.

In some embodiments, the culture compositions described herein may comprise additional starter culture for fermenting the food product.

### Use

In a further aspect, the present invention provides the use of one or more manganese scavengers to inhibit or delay Listeria growth in food products. Manganese scavengers have the effect of making less manganese available in a product for Listeria, thus inhibiting or delaying their growth.

In preferred embodiment, provided herein is the use of one or more bacteria strains and/or a chemical chelating material for inhibiting or delaying growth of Listeria in a food product. The one or more bacteria strains may or may not produce bacteriocin.

Preferably, the food product has a pH higher than 4.3 and/or a water activity of higher than 0.92 and may be a fermented product, a dairy, meat or vegetable product. In more preferred embodiments, provided herein is the use of one or more lactic acid bacteria, preferably of the family Lactobacillaceae and more preferably of the genus Lactobacillus as manganese scavenger, where the bacteria can be selected from the group consisting of Lactobacillus plantarum, Lactobacillus fermentum, Lactobacillus reuteri, Lactobacillus sakei, Lactobacillus brevis, Lactobacillus casei, Lactobacillus paracasei, Lactobacillus salivarius, Lactobacillus alimentarius, Pediococcus acidilactici, Lactobacillus rhamnosus and Lactobacillus kefiri for inhibiting or delaying growth of Listeria in food products. Such manganese scavenger may be selected from the group consisting of a) Lactobacillus rhamnosus DSM 32092, b) Lactobacillus rhamnosus DSM 32666, c) Lactobacillus rhamnosus DSM 23035, d) Lactobacillus paracasei DSM 25612, f) Lactobacillus rhamnosus DSM 24616, g) Lactobacillus rhamnosus DSM 33515 and h) a mutant of a)-g) as mother strain, wherein the mutant maintains at least 75% of anti-listerial activity of mother strain to inhibit the growth of Listeria. It is also preferred that the manganese in the product is reduced to a concentration of below 0.006 ppm, preferably below about 0.005 ppm, below about 0.004 ppm, below about 0.003 ppm, below about 0.002 ppm or below about 0.001 ppm.

In some embodiments, the present application provides use of one or more bacteria strains and/or a chemical chelating material for inhibiting or delaying growth of Listeria in a food product, wherein the bacteria strains are added as a Direct Vat Set (DVS) culture composition to reduce manganese in the food product. Preferably, the bacteria strains comprise a manganese transporter designated as TC #2.A.55 or functional variants thereof.

In some embodiments, the present application provides uses of one or more bacteria strains as the manganese scavenger for inhibiting or delaying growth of Listeria in a food product, wherein the bacteria strains are added as a frozen or freeze dried Direct Vat Set (DVS) culture composition to reduce manganese in the food product such as fermented food product like fermented dairy products. Preferably, the bacteria strains comprise a manganese transporter designated as TC #2.A.55 or functional variants thereof.

In one embodiment, the present application provides uses of one or more bacteria strains of the Lactobacillaceae family, preferably of the Lactobacillus strain, as the manganese scavenger for inhibiting or delaying growth of Listeria in a fermented food product, such as fermented dairy products like cheese, wherein the bacteria strains are added as a Direct Vat Set (DVS) culture composition, preferably frozen or freeze-dried, to reduce manganese in the food product. Preferably, the selected bacteria strain comprises a manganese transporter designated as TC #2.A.55 or functional variants thereof and, optionally, does not produce bacteriocin.

In some embodiments, the present application provides uses of one or more bacteria strains of the Lactobacillaceae family, preferably of the Lactobacillus strain, as the manganese scavenger for inhibiting or delaying growth of Listeria in a fermented food product preferably having a pH higher than 4.3 and/or a water activity of higher than 0.92, such as fermented dairy products like cheese, wherein the bacteria strains are added as a Direct Vat Set (DVS) culture composition, preferably frozen or freeze-dried, to reduce manganese in the food product and wherein the bacteria strains comprise a manganese transporter designated as TC #2.A.55 or functional variants thereof and, optionally, does not produce bacteriocin.

In some embodiments, the one or more bacteria strains comprise a manganese transporter having at least 55%, such as at least 60%, 65%, 70%, 75%, 80%, 85%, 90%, 95%, 96%, 97%, 98%, 99% or 100% sequence identity with the sequence of any one of SEQ ID NO: 1-3. In other embodiments, the bacteria strains are free of a superoxide dismutase, preferably free of a manganese superoxide dismutase, and, optionally, measuring a manganese uptake activity of the one or more bacteria strains.

In some embodiments, the present application provides uses of one or more bacteria strains of the Lactobacillaceae family, preferably Lactobacillus plantarum, Lactobacillus fermentum, Lactobacillus reuteri, Lactobacillus sakei, Lactobacillus brevis, Lactobacillus casei, Lactobacillus paracasei, Lactobacillus salivarius, Lactobacillus alimentarius, Pediococcus acidilactici Lactobacillus rhamnosus and Lactobacillus kefiri, as the manganese scavenger for inhibiting or delaying growth of Listeria in a food product, such as fermented dairy products like cheese, wherein the bacteria strains are of the Lactobacillaceae family, preferably Lactobacillus plantarum, Lactobacillus fermentum, Lactobacillus reuteri, Lactobacillus sakei, Lactobacillus brevis, Lactobacillus casei, Lactobacillus paracasei, Lactobacillus salivarius, Lactobacillus alimentarius, Pediococcus acidilactici Lactobacillus rhamnosus and Lactobacillus kefiri. Preferably, the bacteria are added as a Direct Vat Set (DVS) culture composition, preferably frozen or freeze-dried, to reduce manganese in the food product. Preferably, the bacteria strain comprises a manganese transporter designated as TC #2.A.55 or functional variants thereof and, optionally, does not produce bacteriocin.

In some embodiments, the present application provides uses of one or more bacteria strains of the Lactobacillus genus, preferably Lactobacillus plantarum, Lactobacillus fermentum, Lactobacillus reuteri, Lactobacillus sakei, Lactobacillus brevis, Lactobacillus casei, Lactobacillus paracasei, Lactobacillus salivarius, Lactobacillus alimentarius, Lactobacillus rhamnosus and Lactobacillus kefiri, as the manganese scavenger, for inhibiting or delaying growth of Listeria in a fermented dairy products like cheese, such as neutral cheese.

In some embodiments, the present application provides uses of at least two bacteria strains of the Lactobacillus genus, preferably selected from the group consisting of Lactobacillus plantarum, Lactobacillus fermenturn, Lactobacillus reuteri, Lactobacillus sakei, Lactobacillus brevis, Lactobacillus casei, Lactobacillus paracasei, Lactobacillus salivarius, Lactobacillus alimentarius, Lactobacillus rhamnosus and Lactobacillus kefiri, as the manganese scavenger, for inhibiting or delaying growth of Listeria in a fermented dairy products like cheese, wherein, optionally, the bacteria srains are added as a Direct Vat Set (DVS) culture composition, preferably frozen or freeze-dried, to reduce manganese in the food product.

In the such uses, the manganese in the product may be reduced to a concentration of below 0.006 ppm, preferably below about 0.005 ppm, below about 0.004 ppm, below about 0.003 ppm, below about 0.002 ppm or below about 0.001 ppm. In some embodiments, the culture compositions described herein may comprise additional starter culture for fermenting the food product.

### Composition

The manganese scavenging bacteria can be added as a culture composition, preferably present in a frozen, dried or freeze-dried form, e.g. as a Direct Vat Set (DVS) culture. However, as used herein the culture may also be a liquid that is obtained after suspension of the frozen, dried or freeze-dried cell concentrates in a liquid medium such as water or PBS buffer. Where the culture is a suspension, the concentration of viable cells is in the range of 10⁴ to 10¹² cfu (colony forming units) per ml of the composition including at least 10⁴ cfu per ml of the composition, such as at least 10⁵ cfu/ml, e.g. at least 10⁶ cfu/ml, such as at least 10⁷ cfu/ml, e.g. at least 10⁸ cfu/ml, such as at least 10⁹ cfu/ml, e.g. at least 10¹⁰ cfu/ml, such as at least 10¹¹ cfu/ml. In preparing such compositions, high level of manganese should be avoided, because the bacteria may become less effective in inhibiting or delaying Listeria growth when applied in the food product later. Preferably, the composition comprises up to 600 ppm of manganese and wherein the concentration of the lactic acid bacteria colony forming unit/g of is of at least 1E+10. In preferred embodiments, such products comprises 10-600 ppm of manganese, 30-600 ppm of manganese, 35-600 ppm of manganese, 40-600 ppm of manganese, 45-600 ppm of manganese, 50-600 ppm of manganese, 60-550 ppm of manganese, 100-500 ppm of manganese, 150-450 ppm of manganese, 190-400 ppm of manganese, 200-350 ppm of manganese, 250-300 ppm of manganese.

The composition may additionally comprise cryoprotectants, lyoprotectants, antioxidants, nutrients, fillers, flavorants or mixtures thereof. The composition may be in frozen or freeze-dried form. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectantare known to a skilled person in the art. Suitable cryoprotectants or lyoprotectants include mono-, di-, tri-and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

### Products

In some embodiments, the product is a food product. “Food product” has the common meaning of these terms. “Food product” refers to any food or feed products suitable for consumption by humans or animals. Food products can be fresh or perishable food products as well as stored or processed food products. Food products include, but are not limited to, fruits and vegetables including derived products, grain and grain-derived products, dairy products, meat, poultry and seafood. More preferably, the food product is a meat product, vegetable product or dairy products, such as yogurt, tvarog, sour cream, cheese and the like.

“Cheese product” is a term defined in accordance with relevant official regulations. The standards for such products are well known in the field. According to the Codex Alimentarius, cheeses can be classified using a texture-based classification, established according to the percentage of moisture on a fat-free basis (MFFB). A decrease in MFFB results in a distinction between soft, semisoft, semihard and hard cheeses. Such cheeses are prepared with a ripening step.

The method as disclosed herein are especially applicable to cheese products with high pH (>4.3) and/or high water activity (>0.92). This includes fresh cheese, soft cheese, semisoft cheese, and a few types of hard and semihard cheeses.

In preferred embodiments, the dairy product of the present application is soft or semisoft cheese. In a further embodiment, the cheese is cottage cheese, such as warm-filled cottage cheese. Such cheese is characterized by higher packaging temperatures and longer cooling times (for example cooling from about 13° C. to 7° C. for 72 hours). Contamination is more likely to occur during filling from filling equipment operating at higher temperature.

It should be noted that within the context of the present invention, the term “product” and “food product” in the present invention does not refer to water as such. Although manganese is essential to human nutrition, in water it is generally regarded as unhealthy for humans according to United States Environmental Protection Agency (EPA). Therefore, the treatment of drinking water or waste water to remove excess manganese is sometimes carried out for decontamination and health purposes, which is not related to the spirit of the present invention.

The present invention is especially applicable for food products having intermediate to high water activity. Water activities (aw) determine viability and functionality of microorganisms. Water activity or aw is the partial vapor pressure of water in a substance divided by the standard state partial vapor pressure of water. In the field of food science, the standard state is most often defined as the partial vapor pressure of pure water at the same temperature. Using this particular definition, pure distilled water has a water activity of exactly 1.

The main food categories prone to Listeria contamination are dairy products having intermediate to high water activity, such as yogurt, cream, butter, cheese and the like. However, it is also envisioned that the present invention is suitable for food products having lower water activities, such processed meat, cereals, nuts, spices, dried milk, dried meats and fermented meats.

In preferred embodiment, the product where the methods disclosed in the present invention can be applied is a food product having a water activity (aw) of less than 0.98, such as less than about 0.97, less than about 0.96, less than about 0.95, less than about 0.94, less than about 0.93, less than about 0.92, less than about 0.91, less than about 0.90, less than about 0.89, less than about 0.88, less than about 0.87, less than about 0.86, less than about 0.85, less than about 0.84, less than about 0.83, less than about 0.82, less than about 0.81, less than about 0.80, less than about 0.79, less than about 0.78, less than about 0.77, less than about 0.76, less than about 0.75, less than about 0.74, less than about 0.73, less than about 0.72, less than about 0.71, less than about 0.70 or lower.

In some embodiments, the product is one having a water activity (aw) of about 0.70 to about 0.98, such as about 0.75 to about 0.97, such as about 0.80 to about 0.96, such as about 0.85 to about 0.95.

Methods for measuring water activity are known in the art, for example, as described in Fontana Jr, Anthony J. “Measurement of water activity, moisture sorption isotherms, and moisture content of foods.” Water activity in foods: Fundamentals and applications (2007): 155-173.

The methods of the present invention can be used for manufacturing many types of dairy products, such as yoghurt or cheese. Cheeses are of particular focus because they are susceptible to Listeria contamination. The methods disclosed herein can be used for the manufacturing of cheese products with high pH (>4.3) and/or high water activity (>0.92). This includes fresh cheese, soft cheese, semisoft cheese, and a few types of hard and semihard cheeses.

“Cheese” refers to a product prepared by contacting milk, optionally acidified milk, such as milk that is acidified e.g. by means of a lactic acid bacterial culture and optionally with a coagulant and draining the resultant curd. The term “cheese” includes any form of cheese, such as natural cheese, cheese analogs, cheese (processed cheese). A person skilled in the art knows how to convert the coagulum, also known as curd, into cheese, methods can be found in the literature, see e.g. Kosikowski, F. V., and V. V. Mistry, “Cheese and Fermented Milk Foods”, 1997, 3rd Ed . F. V. Kosikowski, L. L. C. Westport, CT.

In preferred embodiments, the food product has a pH which is higher than 4.3 but lower than 7.0, such as higher than 4.4, such as higher than 4.5, such as higher than 4.6, such as higher than 4.7, such as higher than 4.8, such as higher than 4.9, such as higher than 5.0, such as higher than 5.1, such as higher than 5.2, such as higher than 5.3, such as higher than 5.4, such as higher than 5.5, such as higher than 5.6, such as higher than 5.7, such as higher than 5.8, such as higher than 5.9, such as higher than 6.0, such as higher than 6.1, such as higher than 6.2, such as higher than 6.3, such as higher than 6.4, such as higher than 6.5, such as higher than 6.6, such as higher than 6.7.

With their low pH, some dairy products like most yoghurt or fermented food products are less prone to listerial contamination. However, a potential health hazard could arise if a sufficiently high amount of L. monocytogenes recontaminates milk after heat treatment in small plants where unsophisticated methods are used.

In preferred embodiments, the methods of the present invention are suitable for inhibiting the growth of Listeria during the production and the shelf life of cheeses such as soft and semisoft cheese. Preferred cheeses include cottage cheese, white brined cheese, rindless soft cheese, white mold soft cheese, smear-ripened soft cheese, blue-veined soft cheese and pasta filata cheese. Cottage cheese is particularly preferred.

In one embodiment, the steps described herein are carried out to inhibit or delay growth of Listeria in fermented food products. Fermented food products are foods produced or preserved by the action of microorganisms. Fermentation means the conversion of carbohydrates into alcohols or acids through the action of a microorganism.

In one embodiment, the food product is a product of lactic acid fermentation, i.e. prepared by lactic acid bacteria (LAB) fermentation. “Lactic acid bacterium” designates a gram-positive, microaerophilic or anaerobic bacterium, which ferments sugars with the production of acids including lactic acid as the predominantly produced acid. The food product typically has a pH of about 3.5 to about 6.5, such as about 4 to about 6, such as about 4.5 to about 5.5, such as about 5.

The present invention is particularly useful in inhibiting or delaying growth of Listeria in dairy products. In such products, contamination with Listeria are common and poses the safety risk to consumption of such products. “Dairy product” includes, in addition to milk, products derived from milk, such as cream, ice cream, butter, cheese and yogurt, as well as secondary products such as lactoserum and casein and any prepared food containing milk or milk constituents as the main ingredient, such as formula milk. In one preferred embodiment, the dairy product is a fermented dairy product.

The term “milk” is to be understood as the lacteal secretion obtained by milking of any mammal, such as cows, sheep, goats, buffaloes or camels. Milk base can be obtained from any raw and/or processed milk material as well as from reconstituted milk powder. Milk base can also be plant-based, i.e. prepared from plant material e.g. soy milk, almond milk, cashew milk or coconut milk. Milk base prepared from milk or milk components from cows is preferred. In some preferred embodiments, the milk is raw milk (i.e. unpasteurized) obtained from cows, sheep, goats, buffaloes or camels.

Concentration of manganese varies in milk, depending on the animal from which it is produced, the feed, as well as the season. In general, manganese is present at a concentration of at least 0.03 ppm in dairy products, for example at least 0.08 ppm for skimmed milk, and at least 0.1 ppm for whole milk. With the present finding of the inventors, reducing the manganese amount in such products or products prepared therefrom would render them more resistant to spoilage.

In one embodiment, the food product is a product prepared by fermentation with thermophiles, i.e. thermophilic fermented food product. The term “thermophile” refers to microorganisms that thrive best at temperatures above 43° C. The industrially most useful thermophilic bacteria include Streptococcus spp. and Lactobacillus spp. The term “thermophilic fermentation” herein refers to fermentation at a temperature above about 35° C., such as between about 35° C. and about 45° C. “Thermophilic fermented food product” refers to fermented food products prepared by thermophilic fermentation of a thermophilic starter culture. Include in such products are for example yogurt, skyr, labneh, lassi, ayran and doogh.

In one embodiment, the food product is a product prepared by fermentation with mesophiles, i.e. mesophilic fermented food product. The term “mesophile” refers to microorganisms that thrive best at moderate temperatures (15° C-40° C.). The industrially most useful mesophilic bacteria include Lactococcus spp. and Leuconostoc spp. The term “mesophilic fermentation” herein refers to fermentation at a temperature between about 22° C. and about 35° C. “Mesophilic fermented food product,” which refers to fermented food products prepared by mesophilic fermentation of a mesophilic starter culture. Included in such products are for example buttermilk, sour milk, cultured milk, smetana, sour cream and fresh cheese, such as quark, tvarog and cream cheese.

The methods disclosed herein are particularly useful to inhibit or delay Listeria growth in fermented milk product such as thermophilic and mesophilic fermented milk product, for example a yogurt product. The term “fermented milk product” is a term generally defined in accordance with relevant official regulations and the standards are well known in the field. For example, symbiotic cultures of Streptococcus thermophilus and Lactobacillus delbrueckii subsp. bulgaricus are used as starter culture for yogurt, whereas Lactobacillus acidophilus is used to make acidophilus milk. Other mesophilic lactic acid bacteria are used to produce quark or fromage frais.

The expression “fermented milk product” means a food or feed product wherein the preparation of the food or feed product involves fermentation of a milk base with a lactic acid bacterium. “Fermented milk product” as used herein includes but is not limited to products such as thermophilic fermented milk products (e.g. yogurt) and mesophilic fermented milk products (e.g. sour cream and buttermilk, as well as fermented whey, quark and fromage frais). Fermented milk product also includes cheese, such as continental type cheese, fresh cheese, soft cheese, cheddar, mascarpone, pasta filata, mozzarella, provolone, white brine cheese, pizza cheese, feta, brie, camembert, cottage cheese, Edam, Gouda, Tilsiter, Havarti or Emmental, Swiss cheese, and Maasdammer.

During food processing chemical preservatives have traditionally been used to avoid Listeria contamination. However, in view of a strong societal demand for less processed and preservative-free foods, the invention contributes to provide an effective solution to manage Listeria growth by using biological manganese scavengers to reduce the manganese concentration.

When using a biological scavenger, the skilled person is able to adjust various parameters such as pH, temperature, and amount of manganese scavenger or bacteria to achieve the desired results, taking into consideration the examples provided in this invention as well as the properties of the food product such as water activity, nutrients, level of naturally occurring manganese, shelf life, storage conditions, packing, etc.

The product in which manganese concentration is reduced is preferably packaged to further limit contact with Listeria. It is also preferred to store the product under cold temperature (below 15° C.) to help inhibit Listeria growth.

For fermented food product, manganese scavenging bacteria may be added before, at the start, or during the fermentation. Depending on parameters chosen, the step of reducing manganese level to a preferred level may take several hours, such as at least 5 hours, such as at least 10 hours, such as at least 15 hours, such as at least 20 hours, such as at least 1 day, 2 days, 3 days or more. A skilled person in the art will be able to choose appropriate parameters, depending on the product where inhibition or delay of Listeria is desired.

The invention provides a method of preparing a fermented food product, comprising adding a starter culture and a manganese scavenger to a food substrate, fermenting the substrate for a period of time until a target pH is reached. The manganese scavenger is preferably a Lactobacillus bacteria strain.

As used herein, the term “food substrate” base refers to the substrate in which fermentation is to be carried out.

To make fermented dairy products, the food substrate is a milk base. Milk base is broadly used in the present invention to refer to a composition based on milk or milk components which can be used as a medium for growth and fermentation of a starter culture.

Milk bases include, but are not limited to, solutions/suspensions of any milk or milk like products comprising protein, such as whole or low-fat milk, skim milk, buttermilk, reconstituted milk powder, condensed milk, dried milk.

Milk base may also be lactose-reduced depending on the need of the consumers. Lactose-reduced milk can be produced according to any method known in the art, including hydrolyzing the lactose by lactase enzyme to glucose and galactose, or by nanofiltration, electrodialysis, ion exchange chromatography and centrifugation.

To ferment the milk base a starter culture is added. The term “starter” or “starter culture” as used in the present context refers to a culture of one or more food-grade microorganisms in particular lactic acid bacteria, which are responsible for the acidification of the milk base.

The manganese scavenger can be added before, at the start, or during the fermentation at the same time or at a different time with the starter culture.

After adding the starter culture and the manganese scavengers and subjecting the milk base to a suitable condition, the fermentation process begins and continues for a period of time. A person of ordinary skill in the art knows how to select suitable process conditions, such as temperature, oxygen, addition of carbohydrates, amount and characteristics of microorganism(s) and the process time it takes. This process may take from three, four, five, six hours or longer.

These conditions include the setting of a temperature which is suitable for the particular starter culture strains. For example, when the starter culture comprises mesophilic lactic bacteria, the temperature can be set to about 30° C., and if the culture comprises thermophilic lactic acid bacterial strains, the temperature is kept in the range of about 35° C. to 50° C., such as 40° C. to 45° C. The setting of the fermentation temperature also depends on the enzyme(s) such as coagulants added to the fermentation which can be readily determined by a person of ordinary skill in the art. In a particular embodiment of the invention the fermentation temperature is between 35° C. and 45° C., preferably between 37° C. and 43° C., and more preferably between 40° C. and 43° C. In another embodiment, the fermentation temperature is between 15° C. and 35° C., preferably between 20° C. and 35° C., and more preferably between 30° C. and 35° C.

Fermentation can be terminated using any methods known to in the art. In general, depending on various parameters of the process, the fermentation can be terminated by making the milk base unsuitable for the strain(s) of the starter culture to grow. For example, termination can be carried out by rapid cooling of the fermented milk product when a target pH is reached. It is known that during fermentation acidification occurs, which leads to the formation of a three-dimensional network consisting of clusters and chains of caseins. The term “target pH” means the pH at which the fermentation step ends. The target pH depends on the fermented milk product to be obtained and can be readily determined by a person of ordinary skill in the art.

In a particular embodiment of the invention, fermentation is carried out until at least a pH of 5.2 is reached, such as until a pH of 5.1, 5.0, 4.9, 4.8, 4.7, 4.6, 4.5, 4.4, 4.3, 4.2, 4.1, 4.0, 3.9, 3.8 or 3.7 is reached. Preferably, the fermentation is carried out until a target pH between 4.0 and 5.0 and more preferably between 4.0 and 4.6 is reached. In a preferred embodiment, the fermentation is carried out until target pH below 4.6 is reached.

In a preferred embodiment, fermented food product is selected from the group consisting of quark, cream cheese, fromage frais, greek yogurt, skyr, labneh, butter milk, sour cream, sour milk, cultured milk, kefir, lassi, ayran, twarog, doogh, smetana, yakult and dahi.

In another preferred embodiment, fermented food product is a cheese, including continental type cheese, fresh cheese, soft cheese, cheddar, mascarpone, pasta filata, mozzarella, provolone, white brine cheese, pizza cheese, feta, brie, camembert, cottage cheese, Edam, Gouda, Tilsiter, Havarti or Emmental, Swiss cheese, and Maasdamer.

In a further embodiment, the method further comprises packing the food product to reduce contact with Listeria.

Included in the present invention is a food product obtained by the methods described herein.

The product obtained by the present invention is preferably a fermented milk product with a concentration of manganese reduced to less than 0.006 ppm after being stored for at least two days, for example at least 3 days, at least 4 days, more preferably at least 5 days, at least 6 days, at least 7 days, at least 8 days, at least 9 days, at least 10 days, at least 11 days, at least 12 days, at least 13 days, and at least 14 days.

The manganese scavenging bacteria can be Lactobacillus rhamnosus or Lactobacillus paracasei.

In a preferred embodiment, the manganese scavenging bacteria is Lactobacillus rhamnosus DSM 32092 or a mutant of Lactobacillus rhamnosus DSM 32092, wherein the mutant maintains at least 75% of the anti-listerial activity of Lactobacillus rhamnosus DSM 32092 to inhibit the growth of Listeria. Inhibition may be determined according to the assay as described herein.

In a preferred embodiment, the manganese scavenging bacteria is Lactobacillus rhamnosus DSM 32666 or a mutant of Lactobacillus rhamnosus DSM 32666, wherein the mutant maintains at least 75% of the anti-listerial activity of Lactobacillus rhamnosus DSM 32666 to inhibit the growth of Listeria. Inhibition may be determined according to the assay as described herein.

In a preferred embodiment, the manganese scavenging bacteria is Lactobacillus rhamnosus DSM 23035 or a mutant of Lactobacillus rhamnosus DSM 23035, wherein the mutant maintains at least 75% of the anti-listerial activity of Lactobacillus rhamnosus DSM 23035 to inhibit the growth of Listeria. Inhibition may be determined according to the assay as described herein.

In a preferred embodiment, the manganese scavenging bacteria is Lactobacillus paracasei DSM 25612 or a mutant of Lactobacillus paracasei DSM 25612, wherein the mutant maintains at least 75% of the anti-listerial activity of Lactobacillus paracasei DSM 25612 to inhibit the growth of Listeria. Inhibition may be determined according to the assay as described herein.

In a preferred embodiment, the manganese scavenging bacteria is Lactobacillus rhamnosus DSM 24616 or a mutant of Lactobacillus rhamnosus DSM 24616, wherein the mutant maintains at least 75% of the anti-listerial activity of Lactobacillus rhamnosus DSM 24616 to inhibit the growth of Listeria. Inhibition may be determined according to the assay as described herein.

In a preferred embodiment, the manganese scavenging bacteria is Lactobacillus rhamnosus DSM 33515 or a mutant of Lactobacillus rhamnosus DSM 33515, wherein the mutant maintains at least 75% of the anti-listerial activity of Lactobacillus rhamnosus DSM 33515 to inhibit the growth of Listeria. Inhibition may be determined according to the assay as described herein.

In the present context, the term “mutant” should be understood as a strain derived from a strain of the invention by means of e.g. genetic engineering, radiation and/or chemical treatment. It is preferred that the mutant is a functionally equivalent mutant, e.g. a mutant that has substantially the same, or improved, properties (e.g. regarding anti-Listeria properties) as the mother strain. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the bacterial genome have been shifted with another nucleotide, or deleted, compared to the mother strain.

### DETERMINATION OF ANTI-LISTERIAL ACTIVITY (ANTI-LISTERIAL ASSAY)

The assay for determining anti-listerial activity in dairy product can for example be performed in a cottage cheese model using the following steps:

Heat-treat skim milk (0.1% fat, 3.6% protein) at 90° C. for 5 minutes. Inoculate 200 ml treated milk simultaneously with 0.029% (w/w) starter culture of Lactococcus lactis subsp. lactis and Streptococcus thermophilus (Fresco 1000NG-10, Chr. Hansen A/S, Denmark) and 2×10⁷ CFU/mL of the strain to be determined.

Ferment the inoculated milk at 35° C. until reaching pH 4.65. Afterwards, place samples in a water bath at 57° C. for 90 minutes. Centrifuge at 500 g for 3 minutes and remove supernatant to obtain a curd. Cool the curd to 12-13° C. and store at 13° C. for later mixing with dressing.

To make the dressing, heat-treat cream (10.5% fat, no added salt) at 90° C. for 5 minutes to ensure low background flora. Inoculate the heat-treated cream with a mix of three L. monocytogenes strains in equal amounts:
mhl210 (obtainable from the Copenhagen University from Department of Veterinary and Animal Sciences, Section for Food Safety and Zoonoses),
ATCC 13932 (obtainable from ATCC) and
DSM 15675 (obtainable from DSMZ).

Purify each strain using Listeria selective PALCAM agar (Oxoid®, Thermo Fisher Scientific, Waltham, MA). Take a loop of material using an inoculation loop and transferred to a tube containing 10 mL PALCAM media. Incubate the tube at 30° C. overnight. Transfer 200 μL of the inoculum to 200 mL B-milk (ISO 26323:2009) and grow overnight at 30° C. to acclimate the Listeria strains to milk environment.

Inoculate the dressing with the mixture of Listeria monocytogenes. Mix the dressing with the curd (ratio 60:40% (w/w)) to give a final Listeria concentration of 1×10³ CFU/g. Store the sample at 7° C.

Sample 5 mL of cottage cheese on the sampling day and add it to a stomacher bag with 45 mL demineralized water. Stomach the sample until homogenized prepare a dilution row from 10⁻¹ to 10⁻⁶-band plate on Listeria-selective PALCAM agar plates (Oxoid® CM877, Thermo Fisher Scientific, Waltham, MA). Incubate the plates at 30° C. for 2-3 days for enumeration. The colonies are grey-green in color with a black halo against the red medium background.

Other features and advantages of the invention will become apparent from reading the following description in conjunction with the accompanying figures. Unless otherwise defined, all terms used herein have the same meaning as commonly understood by one of ordinary skill in the art. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. Unless otherwise stated, all exact values provided herein are representative of corresponding approximate values (e.g. all exact exemplary values provided with respect to a particular factor or measurement can be considered to also provide a corresponding approximate measurement, modified by “about”, where appropriate). The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DEPOSIT AND EXPERT SOLUTION

The applicant requests that a sample of the deposited microorganisms stated below may only be made available to an expert, until the date on which the patent is granted.

The applicant deposited the Lactobacillus rhamnosus DSM 32092 on 2015 Jul. 16 at Leibniz Institute DSMZ—German Collection of Microorganisms and Cell Cultures, Inhoffenstr. 7B, D-38124 Braunschweig and received the accession No.: DSM 32092.

The applicant deposited the Lactobacillus rhamnosus DSM 32666 on 2017 Oct. 17 at Leibniz Institute DSMZ—German Collection of Microorganisms and Cell Cultures, Inhoffenstr. 7B, D-38124 Braunschweig and received the accession No.: DSM 32666.

The applicant deposited the Lactobacillus rhamnosus DSM 23035 on 2009 Oct. 14 at Leibniz Institute DSMZ—German Collection of Microorganisms and Cell Cultures, Inhoffenstr. 7B, D-38124 Braunschweig and received the accession No.: DSM 23035.

The applicant deposited the Lactobacillus rhamnosus DSM 25612 on 2012 Feb. 2 at Leibniz Institute DSMZ—German Collection of Microorganisms and Cell Cultures, Inhoffenstr. 7B, D-38124 Braunschweig and received the accession No.: DSM 25612.

The applicant deposited the Lactobacillus rhamnosus DSM 24616 on 2011 Mar. 1 at Leibniz Institute DSMZ—German Collection of Microorganisms and Cell Cultures, Inhoffenstr. 7B, D-38124 Braunschweig and received the accession No.: DSM 24616.

The applicant deposited the Lactobacillus rhamnosus DSM 33515 on 2020 May 5 at Leibniz Institute DSMZ—German Collection of Microorganisms and Cell Cultures, Inhoffenstr. 7B, D-38124 Braunschweig and received the accession No.: DSM 33515.

### EXAMPLES

### Example 1 Inhibition of Listeria monocytogenes and Listeria innocua

This example illustrates the effect of manganese depletion on growth of monocultures of Listeria spp. in a chemically defined medium (CDM). Lactococcus lactis subsp. cremoris MG1363 was included as comparison.

### Culture Medium

The composition of the used CDM is provided in Table 4. All the components were dissolved in 700 mL Milli-Q water to minimize traces of manganese, and the pH was set to 6.8 with KOH, after which the volume was adjusted to 1 L and filter-sterilized. With the presence of manganese, this CDM supports growth of both Listeria spp. and L. lactis.

TABLE 4
Composition of manganese-deficient
chemically defined medium (CDM)

| | Final | | | Final | |
|---|---|---|---|---|---|
| Component | concentration | | Component | concentration | |
| L-Alanine | 3.4 | mM | Niacinamide | 8.2 | μM |
| L-Glutaminic | 2.1 | mM | Thiamine HCl | 3.0 | μM |
| acid | | | | | |
| Proline | 2.6 | mM | Ca-Pantothenate | 2.1 | μM |
| L-Serine | 2.8 | mM | Pyridoxal HCl | 9.8 | μM |
| L-Arginine | 1.1 | mM | MgCl₂•6H₂O | 1.0 | mM |
| Glycine | 2.7 | mM | CaCl₂•2H₂O | 0.3 | mM |
| L-Lysine | 1.4 | mM | ZnSO₄•7H₂O | 17.4 | μM |
| L-Phenyl- | 1.2 | mM | CoSO₄•7H₂O | 8.9 | μM |
| alanine | | | | | |
| L-Threonine | 1.7 | mM | CuSO₄•5H₂O | 10 | μM |
| L-Asparagine | 0.8 | mM | (NH₄)6Mo₇O₂₄•4H₂O | 2.0 | μM |
| L-Glutamine | 0.7 | mM | FeCl₃•6H₂O | 5.5 | μM |
| L-Isoleucine | 0.8 | mM | FeCl₂•4H₂O | 12.6 | μM |
| L-Leucine | 0.8 | mM | K₂HPO₄ | 5.7 | mM |
| L-Methionine | 0.7 | mM | KH₂PO₄ | 3.7 | mM |
| L-Tryptophan | 0.5 | mM | NaOAc | 15 | mM |
| L-Valine | 0.9 | mM | (NH₄)3-citrate | 2.5 | mM |
| L-Histidine | 0.3 | mM | L(+)ascorbic acid | 2.8 | mM |
| Biotin | 0.4 | μM | Tyrosine | 0.3 | mM |
| Folic Acid | 2.3 | μM | Cysteine | 0.8 | mM |
| Riboflavin | 2.6 | μM | Lipoic acid | 9.7 | μM |
| L-Aspartic acid | 0.8 | mM | | | |

### Listeria and L. lactis Strains

The following strains were tested:
1. L. monocytogenes 1: DSM 15675 (obtainable from DSMZ)
2. L. monocytogenes 2: mhl210 (obtainable from the Copenhagen University from Department of Veterinary and Animal Sciences, Section for Food Safety and Zoonoses)
3. L. monocytogenes 3: ATCC 13932 (obtainable from ATCC)
4. L. innocua BL 86/26
5. L. lactis subsp. cremoris MG1363 (MoBiTec GmbH, Germany)

### Growth Experiment

L. innocua BL 86/26 and L. lactis MG1363 were pre-grown from -80° C. glycerol stocks in M17 broth with 1% (w/v) glucose. The three L. monocytogenes were pre-grown from −80° C. glycerol stocks in BHI. All cultures were incubated overnight as standing cultures at 30° C., resulting in stationary phase cultures. The next day, 1 mL of each culture was washed twice with and resuspended in manganese-deficient CDM to remove residual manganese. 1 μL of each washed culture was then used to inoculate, in triplicate, a well of a 96-wells plate containing 200 μL CDM supplemented with 1% glucose diluted from a 50% (w/v) stock solution dissolved in demineralized water, in which manganese was left out, or added to a final concentration of 0.00006, 0.0006, 0.006, 0.06 or 0.6 mg L⁻¹ manganese. Growth was followed by monitoring the absorbance at 600 nm at 30° C. in a Synergi H1 reader (BioTek, Winooski, VT, USA). This experiment was repeated twice on separate days to check reproducibility.

### Results

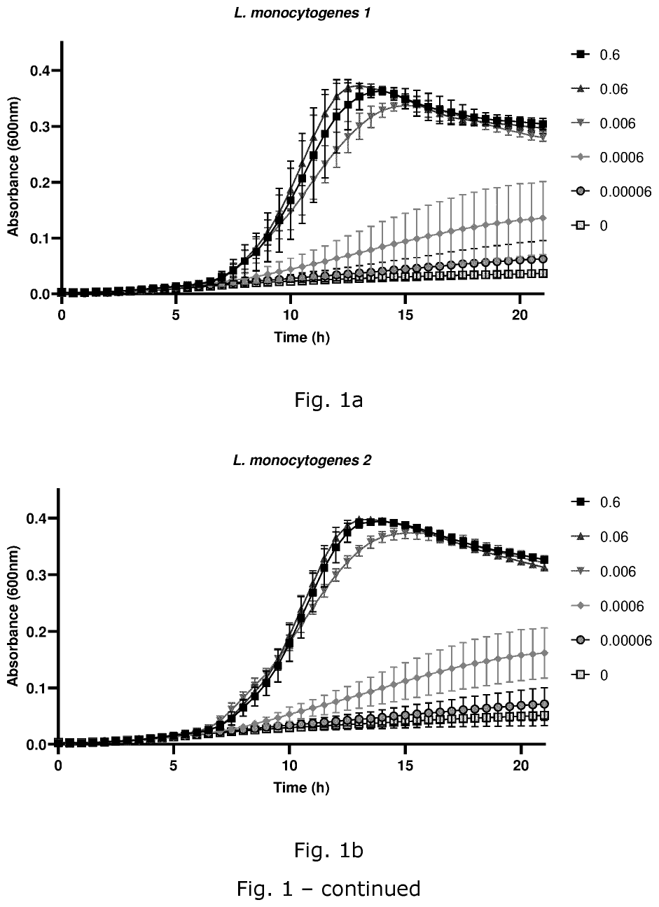

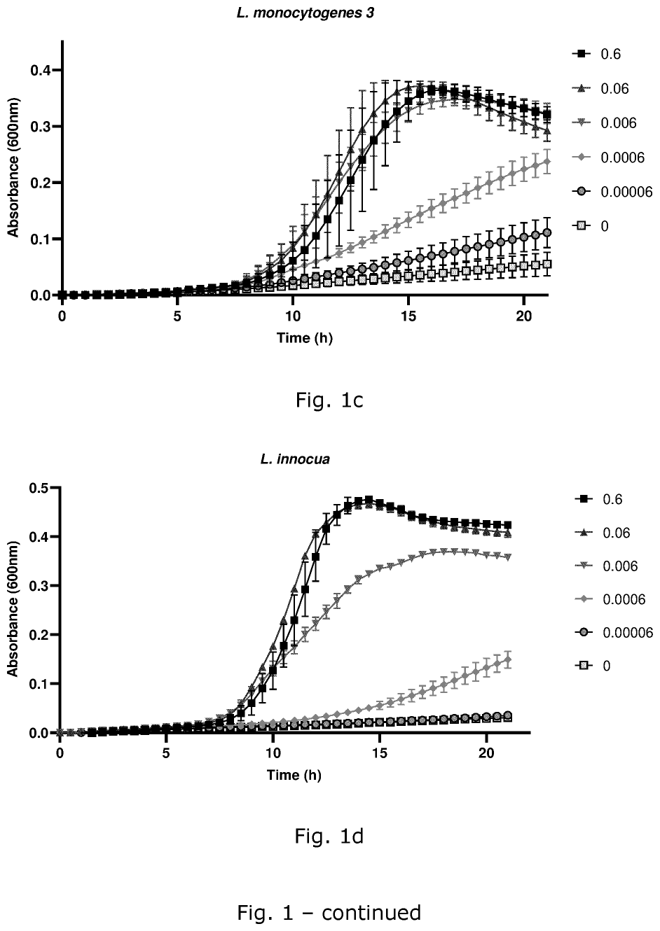

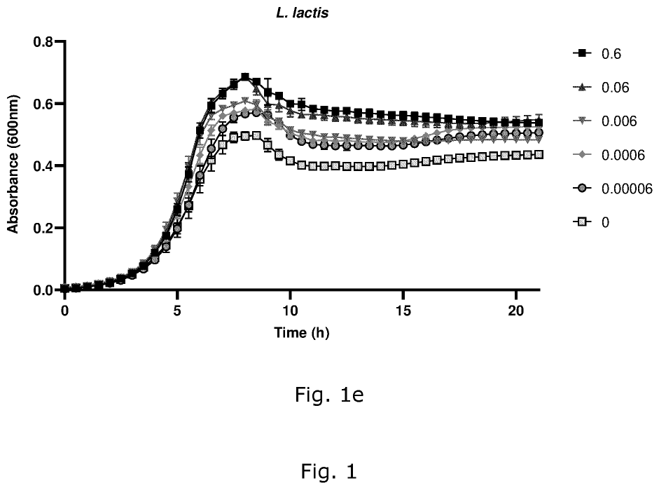

The growth curves of the four Listeria spp. strains and L. lactis subsp.cremoris MG1363 in CDM supplemented with increasing levels of manganese are shown in FIG. 1a-e, respectively. The following manganese concentrations were added to the CDM prior to inoculation: 0.6 ppm (black squares), 0.06 ppm (upright grey triangle), 0.006 ppm (upside down grey triangle), 0.0006 ppm (light grey diamond), 0.00006 ppm (light grey circle), 0 ppm (light grey square).

As shown in FIG. 1a-d, at the concentrations of 0.6 ppm and 0.06 ppm, no growth defect can be observed. However, when manganese was added to a final concentration of 0.006 ppm, some growth defects become apparent. The growth was furthermore severely hampered when 0.0006 ppm manganese was added. The inhibitory effect due to low manganese levels is maintained for at least 20 hours.

Unlike the Listeria strains, growth of L. lactis subsp. cremoris MG1363 was hardly inhibited by the depletion of manganese in the same medium (FIG. 1e).

This example illustrates that manganese depletion has an inhibitory effect on the growth of tested Listeria spp. but not L. lactis subsp. cremoris. Inhibition of Listeria can be seen as soon as the concentration manganese drops below 0.006 ppm. A concentration of above 0.006 ppm restored the growth of Listeria.

### Example 2 Inhibition of Listeria innocua in Cottage Cheese Model Using Manganese Scavenging Bacteria

This example shows the effect of L. rhamnosus DSM 32092 and the addition of manganese on growth of L. innocua in a cottage cheese model.

To follow the growth of L. innocua in a cottage cheese model in real-time, L. innocua BL 86/26 was modified to express the red fluorescence protein (RFP) mCherry only during active growth. Red fluorescence can be detected in fermented milk products and therefore can be used to indicate L. innocua growth.

### Preparation of Curd for Cottage Cheese Model

Skimmed milk was heat-treated at 90° C. for 5 minutes to ensure a low background flora and transferred to 200 mL bottles. The 200 ml milk was inoculated with 0.03% (w/w) starter culture of Lactococcus lactis subsp. lactis and Streptococcus thermophilus (Fresco 1000NG-10, Chr. Hansen A/S, Denmark) or 0.03% (w/w) Fresco 1000NG-10 and 2×10⁷ CFU mL⁻¹ L. rhamnosus DSM 32092. The milk was fermented at 35° C. until a pH of 4.65 was reached. The samples were heat-treated for 90 minutes in a water bath at 57° C. to simulate cottage cheese scalding. The bottles were centrifuged at 500 g for 3 minutes and the supernatant was removed, and a curd was obtained and stored at 4° C. until further use.

### Monitoring Red Fluorescence Produced by L. innocua

To mimic the mixing of curd with a cream filling, which creates a temporary increase in nutrients, temperature and pH, the curd was mixed with sterilized milk in a 1-to-1 ratio (w/v). The pH was re-adjusted to 5.8 with 1N NaOH. The samples were divided, and 6 ppm manganese was added to each half of the samples. A RFP-tagged version of L. innocua BL 86/26 strain was obtained by introducing a pNZ8148 vector (MoBiTec, Goettingen, Germany) carrying the constitutive P11 promoter developed for Lacticaseibacillus plantarum (Rud, I., Jensen, P. R., Naterstad, K., Axelsson, L. (2006) A synthetic promoter library for constitutive gene expression in Lactobacillus plantarum. Microbiology) followed by the mCherry gene (GenScript, Piscataway, NJ, USA). An overnight culture of the RFP-tagged L. innocua strain, grown in M17+0.5% (w/v) glucose+10 μg mL⁻¹ chloramphenicol, was washed twice with, and resuspended in the manganese-deficient CDM described in Example 1 to remove residual manganese. 5 μL of this material was used to inoculate 1 g of each curd-milk mixture. 180 μL of each mixture was then pipetted in multitude in a 96-wells plate and the fluorescence development was followed for 24 hrs at 30° C. in a Synergi H1 reader (BioTek, Winooski, VT, USA). This experiment was repeated twice on separate days to check reproducibility. Note that the pH and the temperature were set to 5.8 and 30° C. to favor growth of L. innocua.

### Monitoring Acidification After Mixing in a Cottage Cheese Model

Curd samples were prepared and mixed with sterilized milk as described in the previous section, but not inoculated with L. innocua. Blue pH indicator dye was added (50 μL mL⁻¹) and 12×180 μL of each mixture was transferred to a low 96-wells plate. The plate was incubated for 24 hrs at 30° C. on a flat-bed scanner and scanned at the bottom using color-of-pH method as described in Poulsen et al. 2019 (Poulsen, V. K., Derkx, P., Øregaard, G. (2019): “High-Throughput Screening for Texturing Lactococcus Strains”. FEMS Microbiological Letters), where color (hue) values were converted to pH values.

### Results

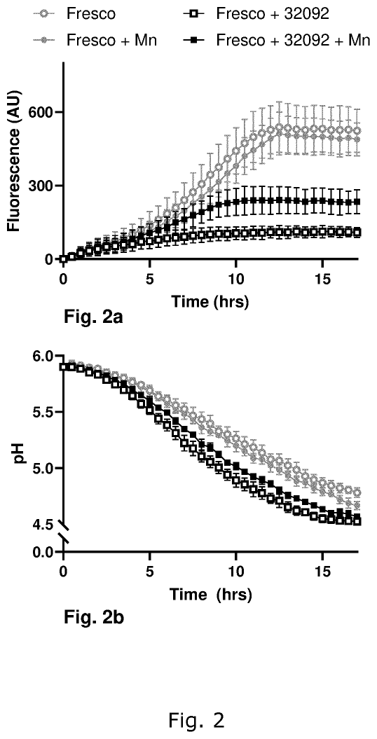

The development of red fluorescence, corresponding to growth of L. innocua, in the model cottage cheese is displayed in FIG. 2a. The aligned acidification curves of the samples not inoculated with L. innocua are shown in FIG. 2b. The cottage cheese model originating from milk fermented with starter culture Fresco 1000NG-10 and the manganese scavenging bacteria L. rhamnosus DSM 32092 (“Fresco+32092”), did not show an increase in the red fluorescence signal, indicating inhibited growth of L. innocua (FIG. 2a). In contrast, the red fluorescent signal increased over time in the samples fermented with only the starter culture (“Fresco”). The addition of 6 ppm manganese resulted in an increase in red fluorescence in the samples fermented with the starter culture and manganese scavenger (“Fresco+32092+Mn”), but not in the Fresco samples (“Fresco+Mn”), indicating that manganese as a limiting factor when L. rhamnosus DSM 32092 was present.

The addition of 6 ppm manganese to the “Fresco+32092” samples did not lead to a full restoration in red fluorescent signal to the levels reached in the “Fresco” and “Fresco+Mn” samples. A comparison of the aligned normalized acidification curves (FIG. 2b) and the fluorescence development of each sample (FIG. 2a) reveals that development of red fluorescence came to a halt whenever a pH of around 5-5.2 was reached, which is non-permissive for the growth of L. innocua. In FIG. 2b it can also be seen that the non-permissive pH for L. innocua growth was reached several hours faster in the “Fresco+32092” than in the “Fresco” samples. In the former condition, L. innocua therefore has a shorter period during which growth was feasible. This correlates to the reduced level of red fluorescence observed in “Fresco+32092+Mn” compared to “Fresco” and “Fresco+Mn” samples.

Together, these data demonstrate that, for a pH value of 5-5.2 and above, the level of manganese constitutes the growth-limiting factor for Listeria. Furthermore, the use of manganese scavenging lactic acid bacteria has the additional effect of rapidly reducing the pH to a level which is non-permissive for Listeria, and as a result limits the time frame for potential Listeria growth.

### Example 3 Inhibition of Listeria innocua in Cottage Cheese Model Using Manganese Scavenging Bacteria

In this example, a cottage cheese model which mimics the condition of warm-filled cottage cheese was used for a quantitative examination of the inhibitory effect of the manganese scavenging bacteria Lactobacillus rhamnosus DSM 32092. The curd was prepared the same way as in Example 2 except that it was later mixed with cream dressing instead of milk. This example resembles more the standard preparation of cottage cheese in USA.

### Preparation of Cottage Cheese Model

Curd for the cottage cheese model was prepared as described in Example 2. In addition, samples were included that were supplemented with 6 ppm manganese prior to fermentation. All fermentations were done in duplicate and the resulting curd was cooled to 12-13° C. and stored at 13° C. for later mixing with the dressing. An overview of the performed fermentations is given in Table 5.

TABLE 5
Experimental design

| | Starter | Manganese | |
|---|---|---|---|
| Sample | culture | Scavenger | Manganese |
| Fresco | Lactococcus lactis subsp. | — | — |
| | lactis Streptococcus | | |
| | thermophilus | | |
| Fresco + Mn | Lactococcus lactis subsp. | — | 6 ppm |
| | lactis Streptococcus | | |
| | thermophilus | | |
| Fresco + 32092 | Lactococcus lactis subsp. | DSM 32092 | — |
| | lactis Streptococcus | | |
| | thermophilus | | |
| Fresco + 32092 + | Lactococcus lactis subsp. | DSM 32092 | 6 ppm |
| Mn | lactis Streptococcus | | |
| | thermophilus | | |

To make the dressing, 9% fat cream was mixed with 2% NaCl. The dressing was heat-treated at 90° C. for 5 minutes to ensure a low background flora and inoculated with L. inoccua after cooling. The dressing containing L. inoccua was then mixed 50:50% (w/w) with the curd, giving a final L. inncoua concentration of 1×10³ CFU g⁻¹. One mL of each cottage cheese was sampled on day 1, 8, 16 and 21 of the experiment, and plated on Listeria-selective PALCAM agar plates. The plates were incubated at 30° C. for 2-3 days after which colony forming unit (CFU) counts were performed.

### Results

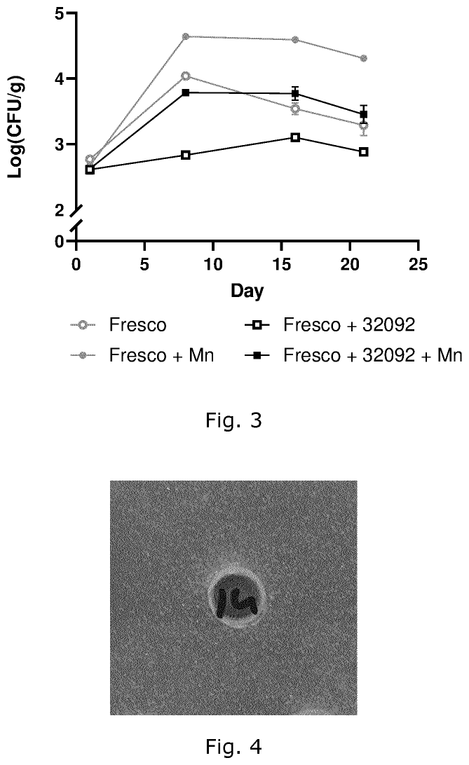

The CFU count of L. innocua in the model cottage cheese obtained at day 1, 8, 16 and 21 is shown in FIG. 3. In alignment with Example 2, the increase in CFU counts was very minor in the sample containing manganese scavenger (“Fresco+32092”) but significant (1 log increase) in samples without (“Fresco”). The addition of 6 ppm manganese at the beginning of the sample containing manganese scavenger (“Fresco+32092+Mn”) resulted in an increase of L. innocua growth, with CFU counts similar to the samples containing the starter culture (“Fresco”).

The supplementation of manganese to the Fresco samples (“Fresco+Mn”) also resulted in an increase in L. innocua CFU counts. This suggests that the low manganese level naturally occurring in milk might already poses limits to the growth of Listeria spp. in dairy products.

### Example 4 Lactobacillus rhamnosus DSM 32092 and Bacteriocin Production

### Well Diffusion Assay

To test if L. rhamnosus DSM 32092 produces anti-listerial bacteriocins, the strain was grown to stationary phase in MRS (de Man, Rogosa, Sharpe) broth placed in an anaerobic jar at 37° C. Supernatant of this culture was collected through centrifugation, which was then filtered using a 0.20 μm filter. The target strain L. innocua BL 86/26 was grown overnight in M17 with 0.5% glucose and subsequently diluted 1000× (v/v) in 0.75% (w/v) M17-based soft agar supplemented with 0.5% glucose. This mixture was poured on top of a 1.5% (w/v) M17 agar leaving out a 10 mm hole. 200 μL of the supernatant was added to the hole after solidification of the agar and the plate was incubated at room temperature for 24 hrs after which pictures of the plates were taken.

### Results

As shown in FIG. 4, no inhibition zone could be observed around the well to which supernatant of L. rhamnosus DSM 32092 was added.

In two separate experiments, well diffusion assays to test for anti-listerial bacteriocin production by L. rhamnosus DSM 32092 were performed using both the curd and the whey collected from the cottage cheese model as described in Example 2, as well as using sterilized milk in which L. rhamnosus DSM 32092 had been growing for 16 hrs. No inhibition zones against L. innocua BL 86/26 could be observed using any of these samples (data not shown).

This indicates that the strain does not secrete antilisterial bacteriocins. Rather, Listeria inhibition is attributed to the manganese scavenging activity of L. rhamnosus DSM 32092.

### Example 5 Growth of Lactococcus lactis and Streptococcus thermophilus Under Manganese Limitation

The performance of the starter culture strains at various manganese concentrations was evaluated. Single Lactococcus lactis and Streptococcus thermophilus strains isolated from the Fresco 1000NG-10 (Chr. Hansen A/S, Denmark), as well as Listeria innocua BL 86/26 as control, were grown in a chemically defined medium (CDM) with different manganese levels.

### Culture Medium

CDM for growth of Lactococcus lactis and Listeria innocua was prepared as described in Example 1. As to Streptococcus thermophilus, a second Mn-free CDM supporting it growth was prepared essentially as described by Otto et al. (“The relation between growth rate and electrochemical proton gradient of Streptococcus cremoris.” FEMS Microbiology Letters 16.1 (1983): 69-74) with the following exceptions: All metals were used in the same final concentrations as listed in Table 4, and all amino acids were added to a final concentration of 0.08 g/L except for cysteine, which consisted of a final concentration of 0.5 g/L. Wolfe's vitamin solution was used in which DL-Ca-pantothenate was increased to have a final concentration of 400 mg/L, and urea and NaHCO₃ were added to a final concentration of 0.12 and 0.42 g, respectively.

### Listeria and L. Lactis Strains

The following strains were tested:
1. L. lactis single strain isolated from Fresco 1000NG-10 (Chr. Hansen A/S, Denmark)
2. S. thermophilus single strain isolated from Fresco 1000NG-10 (Chr. Hansen A/S, Denmark)
3. L. innocua BL 86/26

### Growth Experiment

All strains were pre-grown to stationary phase cultures from −80° C. glycerol stocks in M17 broth with 1% (w/v) glucose, as standing cultures at 30° C. 1 mL of each culture was then washed twice with and resuspended in manganese-deficient CDM to remove residual manganese. 1 μL of each washed culture was then used to inoculate, in triplicate, a well of a 96-wells plate containing 200 μL CDM supplemented with 1% (w/v) glucose diluted from a 50% (w/v) stock solution dissolved in Milli-Q water, and in which manganese was left out, or added to a final concentration of 0.00006, 0.0006, 0.006, 0.06 or 0.6 mg L-1 manganese. Growth was followed by monitoring the absorbance at 600 nm at 30° C. in a Synergi H1 reader (BioTek, Winooski, VT, USA).

### Results

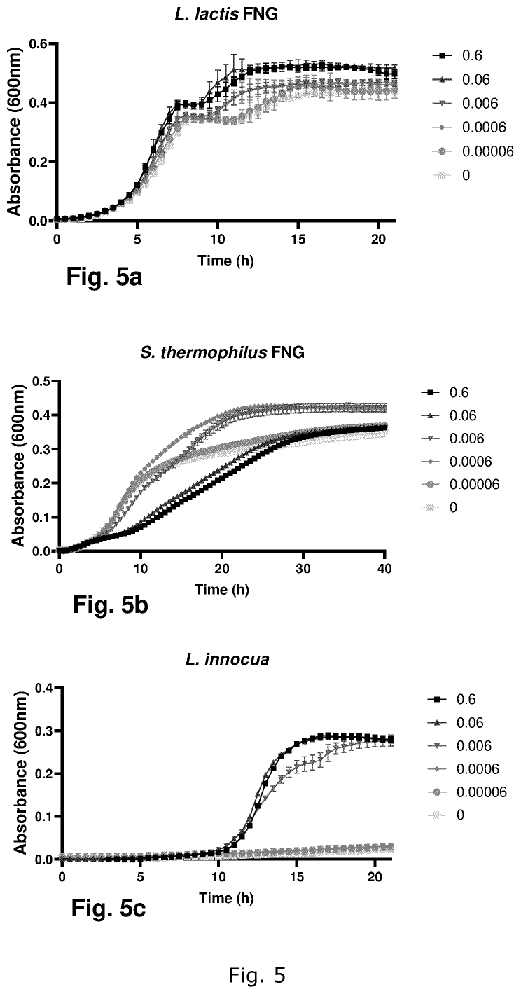

The growth curves of the single L. lactis and S. thermophilus strain as well as L. innocua BL 86/26 in CDM supplemented with various of manganese levels are shown in FIG. 5a-c. The following manganese concentrations were added to the CDM prior to inoculation: 0.6 ppm (black squares), 0.06 ppm (upright grey triangle), 0.006 ppm (upside down grey triangle), 0.0006 ppm (light grey diamond), 0.00006 ppm (light grey circle), 0 ppm (light grey square).

Unlike growth of the Listeria strains shown in Example 1, growth of the L. lactis and S. thermophilus strains from the starter cultures was minimally inhibited by the depletion of manganese (FIG. 5a-b). L. innocua BL 86/26, taken along as a control, required more than 0.0006 ppm manganese for growth to occur (FIG. 5c).

This example illustrates that manganese depletion has no inhibitory effect on the growth of L. lactis or S. thermophilus strains from cheese starter cultures. Low manganese concentration does not adversely affect the growth of starter culture.

### Example 6 Manganese Supplementation and Growth of Listeria monocytogenes in Industrial Cottage Cheese Prepared Using Manganese Scavenging Bacteria

In this example, the growth of milk-adapted L. monocytogenes was followed in warm-filled cottage cheese prepared with or without the presence of manganese scavenging L. rhamnosus DSM 32092. Specifically, the effect of adding 6 ppm manganese during the creaming step was evaluated.

### Listeria Strains

L. monocytogenes mixture consisted of the following strains:
1. L. monocytogenes 1: DSM 15675 (obtainable from DSMZ)
2. L. monocytogenes 2: mhl210 (obtainable from the Copenhagen University from Department of Veterinary and Animal Sciences, Section for Food Safety and Zoonoses)
3. L. monocytogenes 3: ATCC 13932 (obtainable from ATCC)

Here, the Listeria strains were pre-grown in milk to prepare the Listeria for optimal growth in milk. To do so, each strain was first grown in PALCAM Listeria selective broth (Oxoid) from a single colony to early stationary phase at 30° C., diluted 100-fold in standardized boiled milk (B-milk; described in ISO 26323:2009), grown for another 16-hrs at 30° C., and then mixed in equal volumes. The B-milk cultures were frozen and a CFU count was performed after 24-hrs of freezing to calculate the cell concentration of the stocks. Prior to inoculation in cottage cheese, 2 mL of a stock ampoule was dissolved in 100 mL B-milk and used to inoculate the various cottage cheeses to establish the indicated CFU counts.

### Preparation of Cottage Cheese

Skimmed milk was pasteurized, cooled to room temperature, split over 2 vats and mixed with 0.0015% (w/v) CaCl₂ and 0.0001% (v/v) of the microbial coagulant Hannilase XP (Chr. Hansen A/S, Denmark). Vat 1 was inoculated with 0.23 U/L FRESCO 1000NG-20 (Chr. Hansen A/S, Denmark, containing Lactococcus lactis subsp. lactis, Lactococcus lactis subsp. cremoris and Streptococcus thermophilus), and Vat 2 with 0.23 U/L FRESCO 1000NG-20 and 0.1 U/L L. rhamnosus DSM 32092. Both vats were incubated at 33° C. for about 5 h until the coagulum had a pH of 4.74, after which it was cut and incubated at 33° C. for 30 min. During the next step, the cooking process, the temperature was gradually increased over time; from 33° C. to 37° C. for 20 min, 37° C. to 39° C. for 30 min including 1 min stirring every 15 min, 39° C. to 50° C. for 30 min under continuous stirring, from 50° C. to 57° C. for 30 min under continuous stirring. To prepare the cream dressing, 80% (w/w) coffee cream (8.1% fat), 2% (w/w) cream (38.6% fat) and 2% (w/w) salt (NaCl) were blended, homogenized, pasteurized at 90° C. for 10 min, and cooled to 12° C. 0.55 parts of the drained and washed curd was then mixed with 0.45 of cream dressing to create a cottage cheese formulation with a pH of about 5.3. To half of the cottage cheese, 6 ppm manganese was added, and 100-g portions were inoculated with 1×10⁴ CFU/g of milk-adapted L. monocytogenes mixture. The portions stored according to the warmed filled cottage cheese industry cooling profile: 12° C. for 24 h, then stored at 10° C. for 24 h and then at 7° C. for remainder of time. Sampling for L. monocytogenes was performed as written in Example 3 for L. innocua.

### Results

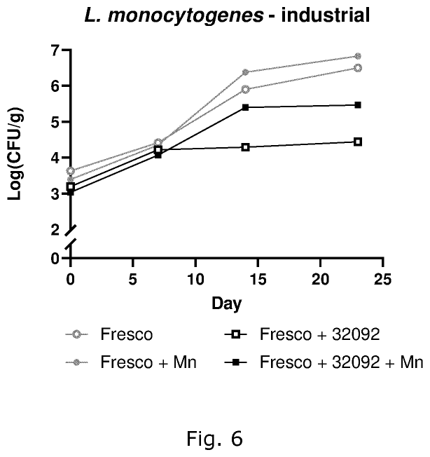

The CFU counts of L. monocytogenes in the cottage cheese formulations obtained at Day 0, 7, 14 and 23 are shown in FIG. 6. All cottage cheese formulation has around 1-log increase of L. monocytogenes at Day 7. After Day 7, populations of L. monocytogenes started to diverge. Lowest Listeria increase can be seen in the cottage cheese prepared with manganese scavenging bacteria (“Fresco+32092”), where the population remained inhibited after day 7. However, when supplemented with 6 ppm manganese, Listeria growth increased (“Fresco+32092+Mn”) and followed a similar trend as the CFU counts in samples to which no manganese and no manganese-scavenging bacteria (“Fresco”) were added. In these samples, Listeria CFUs increased by 2-to-2.5-logs at Day 14 and Day 23 compared to Day 0. When manganese was added to cheese prepared without manganese scavenging bacteria (“Fresco+Mn”), a further increase in Listeria CFU count was observed.

This confirms that manganese scavenging bacteria are able to reduce outgrowth of milk-adapted Listeria in cottage cheese through competitive exclusion of Mn.

1. A method of inhibiting or delaying growth of Listeria in a food product, comprising:
adding to or contacting with the food product one or more manganese scavengers selected from strains of bacteria family Lactobacillaceae and chemical chelating materials,
thereby reducing manganese in the food product.

2. The method according to claim 1, wherein the one or more manganese scavengers comprise one or more strains of bacteria genus Lactobacillus.

3. The method according to claim 2, wherein the one or more strains of bacteria genus Lactobacillus do not produce bacteriocin.

4. The method according to claim 1, wherein the food product has one or both of a pH higher than 4.3 and a water activity higher than 0.92.

5. The method according to claim 1, wherein the food product is a fermented product.

6. The method according to claim 1, wherein the food product is a dairy, meat or vegetable product.

7. The method according to claim 1, wherein the one or more manganese scavengers comprise one or more strains of bacteria family Lactobacillaceae having a manganese transporter selected from manganese transporter TC #2.A.55 and functional variants thereof.

8. The method according to claim 1, wherein the one or more manganese scavengers comprise one or more strains of bacteria family Lactobacillaceae having a manganese transporter having at least 55% sequence identity with the sequence of any one of SEQ ID NOs. 1-3.

9. The method according to claim 1, wherein the one or more manganese scavengers comprise one or more strains of bacteria family Lactobacillaceae that are free of a superoxide dismutase.

10. The method according to any of the preceding claims claim 1, wherein the one or more manganese scavengers comprise one or more strains of one or more species are selected from Lactobacillus plantarum, Lactobacillus fermentum, Lactobacillus reuteri, Lactobacillus sakei, Lactobacillus brevis, Lactobacillus casei, Lactobacillus paracasei, Lactobacillus salivarius, Lactobacillus alimentarius, Pediococcus acidilactici, Lactobacillus rhamnosus and Lactobacillus kefiri.

11. The method according to claim 1, wherein the one or more manganese scavengers comprise one or more strains of one or more species selected from Lactobacillus rhamnosus, Lactobacillus paracasei, and Lactobacillus plantarum.

12. The method according to claim 1, wherein the one or more manganese scavengers comprises one or more anti-listerial strains selected from:
(a) Lactobacillus rhamnosus strain DSM 32092 deposited at Leibniz Institute DSMZ—German Collection of Microorganisms and Cell Cultures (DSMZ) under accession number DSM 32092,
(b) Lactobacillus rhamnosus strain DSM 32666 deposited at DSMZ under accession number DSM 32666,
c) Lactobacillus rhamnosus strain DSM 23035 deposited at DSMZ under accession number DSM 23035,
(d) Lactobacillus paracasei strain DSM 25612 deposited at DSMZ under accession number DSM 25612,
(e) Lactobacillus rhamnosus strain DSM 24616 deposited at DSMZ under accession number DSM 24616,
(f) Lactobacillus rhamnosus strain DSM 33515 deposited at DSMZ under accession number DSM 33515, and
(g) mutants having any one of strains (a)-(g) as a mother strain, wherein the mutant strain exhibits at least 75% of the anti-listerial activity of its mother strain to inhibit growth of Listeria.

13. The method according to claim 1, wherein the step of adding to or contacting with the food product one or more manganese scavengers comprises an ion-exchange chromatography step.

14. The method according to claim 1, wherein the one or more manganese scavengers comprise one or more chemical chelating materials selected from hydrocolloids, ethylenediaminetetraacetic acid, ethylene glycol-bis(β-aminoethyl ether)-N,N,N′,N′-tetraacetic acid, diaminocyclohexanetetraacetic acid, nitrilotriacetic acid, 1,2-bis(o-aminophenoxy)ethane-N,N,N′,N′-tetraacetic acid, and diethylenetriaminepentaacetic acid.

15. The method according to claim 1, wherein the manganese in the product is reduced to a concentration [[of]] below 0.006 ppm.

16. The method according to claim 1, wherein the one or more manganese scavengers comprise one or more strains of bacteria family Lactobacillaceae having manganese transporter TC #2.A.55.

17. The method according to claim 1, wherein the one or more manganese scavengers comprise one or more strains of bacteria family Lactobacillaceae having a manganese transporter having the sequence of any one of SEQ ID NOs. 1-3.

18. The method according to claim 1, wherein the one or more manganese scavengers comprise one or more strains of bacteria family Lactobacillaceae that are free of a manganese superoxide dismutase.

19. The method according to claim 1, wherein the one or more manganese scavengers comprises one or more anti-listerial strains selected from (a) Lactobacillus rhamnosus strain DSM 32092; (b) Lactobacillus rhamnosus strain DSM 32666, (c) Lactobacillus rhamnosus strain DSM 23035, (d) Lactobacillus paracasei strain DSM 25612, (e) Lactobacillus rhamnosus strain DSM 24616, and (f) Lactobacillus rhamnosus strain DSM 33515.

20. The method according to claim 1, wherein the one or more manganese scavengers comprise ethylenediaminetetraacetic acid.
